(19) United States
(12) Patent Application Publication
(10) Pub. No.: US 2024/0011942 A1
(43) Pub. Date: Jan. 11, 2024

(54) SENSOR ELEMENT AND GAS SENSOR

(71) Applicant: NGK INSULATORS, LTD., Nagoya (JP)

(72) Inventors: Takayuki SEKIYA, Nisshin (JP); Yusuke WATANABE, Nagoya (JP); Kodai ICHIKAWA, Nagoya (JP)

(21) Appl. No.: 18/472,553

(22) Filed: Sep. 22, 2023

Related U.S. Application Data
(63) Continuation of application No. PCT/JP2022/014339, filed on Mar. 25, 2022.

(30) Foreign Application Priority Data
Mar. 31, 2021 (JP) 2021-059121

(51) Int. Cl. G01N 27/419; G01N 33/00

(52) U.S. Cl. CPC G01N 27/419; G01N 33/0037

(57) ABSTRACT

A sensor element for detecting a specific gas concentration in a measurement-object gas, the sensor element includes: an element body internally provided with a measurement-object gas flow portion that introduces a measurement-object gas and causes the measurement-object gas to flow therethrough; a reference-gas introduction portion disposed inside the element body, the reference-gas introduction portion being configured to introduce a reference gas; a reference-gas adjustment pump cell having a pump reference electrode disposed inside the element body, the reference-gas adjustment pump cell being configured to pump oxygen into a periphery of the pump reference electrode; and a sensor cell having a voltage reference electrode disposed inside the element body, and a measurement-object gas-side electrode disposed inside or outside the element body, the sensor cell being configured to generate a voltage based on an oxygen concentration in a periphery of the measurement-object gas-side electrode.

### CROSS-REFERENCE TO RELATED APPLICATION

This application is a continuation application of PCT/JP2022/014339, filed on Mar. 25, 2022, which claims the benefit of priority of Japanese Patent Application No. 2021-059121, filed on Mar. 31, 2021, the entire contents of which are incorporated herein by reference.

### BACKGROUND OF THE INVENTION

### 1. Field of the Invention

The present invention relates to a sensor element and a gas sensor.

### 2. Description of the Related Art

Hitherto, a known gas sensor detects the concentration of a specific gas, such as NOx, in a measurement-object gas, such as the exhaust gas of an automobile. For example, Patent Literature 1 describes a gas sensor including an elongate plate-shaped sensor element obtained by stacking a plurality of oxygen-ion-conductive solid electrolyte layers.

A schematic cross-sectional view schematically illustrating an example of the configuration of a gas sensor 900 in such a related art is illustrated in FIG. 13. As illustrated, the gas sensor 900 includes a sensor element 901. The sensor element 901 is an element having a structure in which oxygen-ion-conductive solid electrolyte layers 911 to 916 are stacked. In the sensor element 901, a measurement-object gas flow portion that introduces a measurement-object gas is formed between the lower surface of the solid electrolyte layer 916 and the upper surface of the solid electrolyte layer 914, and the measurement-object gas flow portion is provided with a first internal cavity 920, a second internal cavity 940, and a third internal cavity 961. An inner pump electrode 922 is disposed in the first internal cavity 920, an auxiliary pump electrode 951 is disposed in the second internal cavity 940, and a measurement electrode 944 is disposed in the third internal cavity 961. In addition, an outer pump electrode 923 is disposed on the upper surface of the solid electrolyte layer 916. In contrast, between the upper surface of the solid electrolyte layer 913 and the lower surface of the solid electrolyte layer 914, a reference electrode 942 is disposed which is in contact with a reference gas (e.g., atmospheric gas) serving as a reference for detecting a specific gas concentration in a measurement-object gas. A main pump cell 921 is formed by the inner pump electrode 922, the outer pump electrode 923, and the solid electrolyte layers 914 to 916. A measurement pump cell 941 is formed by the measurement electrode 944, the outer pump electrode 923, and the solid electrolyte layer 914 to 916. A measurement-pump-control oxygen-partial-pressure detection sensor cell 982 is formed by the measurement electrode 944, the reference electrode 942, and the solid electrolyte layers 914, 913. A Vref detection sensor cell 983 is formed by the outer pump electrode 923, the reference electrode 942, and the solid electrolyte layers 913 to 916. A reference-gas adjustment pump cell 990 is formed by the outer pump electrode 923, the reference electrode 942, and the solid electrolyte layers 913 to 916. In the gas sensor 900, when a measurement-object gas is introduced into the measurement-object gas flow portion, oxygen is pumped out or pumped in between the first internal cavity 920 and the outside of the sensor element by the main pump cell 921, and oxygen is further pumped out or pumped in between the second internal cavity 940 and the outside of the sensor element to adjust the oxygen concentration in the measurement-object gas flow portion. NOx in the measurement-object gas after adjustment of the oxygen concentration is reduced in the periphery of the measurement electrode 944. A voltage Vp2 applied to the measurement pump cell 941 is feedback-controlled so that voltage V2 generated in the measurement-pump-control oxygen-partial-pressure detection sensor cell 982 reaches a predetermined target value, thus the measurement pump cell 941 pumps out the oxygen in the periphery of the measurement electrode 944. The NOx concentration in the measurement-object gas is detected based on the pump current Ip2 which flows through the measurement pump cell 941 then. The reference-gas adjustment pump cell 990 pumps oxygen into the periphery of the reference electrode 942 by passing a pump current Ip3 by a voltage Vp3 applied across the reference electrode 942 and the outer pump electrode 923. Thus, when the oxygen concentration of the reference gas in the periphery of the reference electrode 942 decreases, the decrease in the oxygen concentration can be compensated, and reduction in the accuracy of detection of the specific gas concentration is prevented. Furthermore, a voltage Vref is generated between the outer pump electrode 923 and the reference electrode 942 in the Vref detection sensor cell 983. The voltage Vref makes it possible to detect the oxygen concentration in the measurement-object gas outside the sensor element 901.

### CITATION LIST

### Patent Literature

PTL 1: International Publication No. WO2020/004356

### SUMMARY OF THE INVENTION

Meanwhile, at the time of pumping oxygen into the periphery of a reference electrode by flowing a pump current as with the aforementioned reference-gas adjustment pump cell 990, when an oxygen concentration is detected using the voltage of a sensor cell, such as the voltage V2 of the aforementioned measurement-pump-control oxygen-partial-pressure detection sensor cell 982, the accuracy of detection of the oxygen concentration is low in some cases.

The present invention has been made to solve the aforementioned problem, and a main object thereof is to prevent reduction in the accuracy of detection of the oxygen concentration due to a pump current at the time of pumping-in of oxygen, while oxygen is being pumped into the reference-gas introduction portion.

In order to achieve the aforementioned main object, the present invention employs the following solutions.

A sensor element of the present invention is for detecting a specific gas concentration in a measurement-object gas, and includes: an element body including an oxygen-ion-conductive solid electrolyte layer and internally provided with a measurement-object gas flow portion that introduces a measurement-object gas and causes the measurement-object gas to flow therethrough; a reference-gas introduction portion disposed inside the element body, the reference-gas introduction portion being configured to introduce a reference gas serving as a reference for detecting a specific gas concentration in the measurement-object gas; a reference-gas adjustment pump cell having a pump reference electrode disposed inside the element body so as to be in contact with the reference gas introduced to the reference-gas introduction portion, the reference-gas adjustment pump cell being configured to pump oxygen into a periphery of the pump reference electrode; and a sensor cell having a voltage reference electrode disposed inside the element body so as to be in contact with the reference gas introduced to the reference-gas introduction portion, and a measurement-object gas-side electrode disposed inside or outside the element body so as to be in contact with the measurement-object gas, the sensor cell being configured to generate a voltage based on an oxygen concentration in a periphery of the measurement-object gas-side electrode.

The sensor element includes a reference-gas adjustment pump cell, and a sensor cell. The reference-gas adjustment pump cell pumps oxygen into the periphery of the pump reference electrode, thus reduction in the oxygen concentration of the reference gas in the reference-gas introduction portion can be supplemented. In addition, the oxygen concentration in the periphery of the measurement-object gas-side electrode can be detected with the voltage of the sensor cell. In the element body of the sensor element, a pump reference electrode constituting part of the reference-gas adjustment pump cell, and a voltage reference electrode constituting part of the sensor cell are both disposed. In other words, in the sensor element, the pump reference electrode and the voltage reference electrode are separately provided as the electrodes to be in contact with the reference gas in the reference-gas introduction portion. Thus, unlike when one electrode serves as the pump reference electrode as well as the voltage reference electrode (e.g., in the sensor element 901 illustrated in FIG. 13, the measurement electrode 944 serves as the electrode of the measurement pump cell 941 as well as the electrode of the measurement-pump-control oxygen-partial-pressure detection sensor cell 982), at the time of pumping-in of oxygen performed by the reference-gas adjustment pump cell, a pump current does not flow through the voltage reference electrode, thus the voltage of the sensor cell does not include a voltage drop of the voltage reference electrode due to a pump current. Thus, the voltage of the sensor cell has a value which corresponds with higher accuracy to the oxygen concentration in the periphery of the measurement-object gas-side electrode, thus the accuracy of detection of the oxygen concentration using the sensor cell is improved. Based upon the foregoing, in the sensor element, it is possible to prevent reduction in the accuracy of detection of the oxygen concentration due to a pump current at the time of pumping-in of oxygen, while oxygen is being pumped into the reference-gas introduction portion.

In this situation, the reference-gas adjustment pump cell serves as a pumping-in source of oxygen to the periphery of the pump reference electrode, and may have a pumping-in source electrode disposed inside or outside the element body so as to be in contact with the measurement-object gas. In addition, the reference-gas adjustment pump cell may pump out oxygen from the periphery of the pump reference electrode.

The sensor element of the present invention may further include a measurement pump cell that pumps out oxygen produced from the specific gas in a measurement chamber of the measurement-object gas flow portion. The measurement-object gas-side electrode may be a measurement electrode disposed in the measurement chamber, and the sensor cell may be a measurement sensor cell that generates a voltage based on an oxygen concentration in the measurement chamber. In this manner, the pump reference electrode and the voltage reference electrode are separately provided, thus the voltage of the measurement sensor cell has a value which corresponds with higher accuracy to the oxygen concentration in the measurement chamber, and consequently, the accuracy of detection of the oxygen concentration in the measurement chamber using a measurement sensor cell is improved. For example, use of the voltage of the measurement sensor cell to control the measurement pump cell effects on the accuracy of detection of the specific gas concentration in the measurement-object gas. Thus, the accuracy of detection of the specific gas concentration is improved by improving the accuracy of detection of the oxygen concentration in the measurement chamber using a measurement sensor cell.

In this case, the sensor element of the present invention may further include: an adjustment chamber pump cell having an adjustment electrode disposed in an oxygen concentration adjustment chamber upstream of the measurement chamber of the measurement-object gas flow portion, and a pump outer electrode disposed outside the element body, the adjustment chamber pump cell being configured to pump oxygen out from the oxygen concentration adjustment chamber or pump oxygen into the oxygen concentration adjustment chamber; and an outer sensor cell having a voltage outer electrode disposed outside the element body, and the voltage reference electrode, the outer sensor cell being configured to generate a voltage based on an oxygen concentration in the measurement-object gas outside the element body. In the sensor element, the pump outer electrode constituting part of the adjustment chamber pump cell, and the voltage outer electrode constituting part of the outer sensor cell are both disposed outside the element body. In other words, in the sensor element, the pump outer electrode and the voltage outer electrode are separately provided outside the element body. Thus, unlike when one electrode serves as the pump outer electrode as well as the voltage outer electrode (e.g., in the sensor element 901 illustrated in FIG. 13, the outer pump electrode 923 serves as the electrode of the main pump cell 921 as well as the electrode of the Vref detection sensor cell 983), at the time of pumping-out or pumping-in of oxygen performed by the adjustment chamber pump cell, a pump current does not flow through the voltage outer electrode, thus the voltage of the outer sensor cell does not include a voltage drop of the voltage outer electrode due to a pump current. Consequently, the voltage of the outer sensor cell has a value which corresponds with higher accuracy to the oxygen concentration in the measurement-object gas outside the element body, thus the accuracy of detection of the oxygen concentration in the measurement-object gas using an outer sensor cell is improved. As described above, the pump current of the reference-gas adjustment pump cell does not flow through the voltage reference electrode either. Thus, the voltage of the outer sensor cell is the voltage across the voltage outer electrode and the voltage reference electrode, and no pump current flows through each of the voltage outer electrode and the voltage reference electrode. Therefore, the voltage of the outer sensor cell has a value which corresponds with higher accuracy to the oxygen concentration in the measurement-object gas outside the element body.

A first gas sensor of the present invention includes: the sensor element including the aforementioned measurement pump cell and measurement sensor cell; and a measurement pump cell controller that causes the measurement pump cell to pump out oxygen from the measurement chamber by feedback-controlling the measurement pump cell so that the voltage of the measurement sensor cell reaches a target voltage.

In the first gas sensor, as described above, the accuracy of detection of the oxygen concentration in the measurement chamber using a measurement sensor cell of the sensor element has improved, thus the oxygen concentration in the measurement chamber can be adjusted with high accuracy to an oxygen concentration corresponding to a target voltage by feedback-controlling the measurement pump cell so that the voltage of the measurement sensor cell reaches the target voltage. Since the specific gas concentration is detected then based on the pump current which flows through the measurement pump cell, thus the accuracy of detection of the specific gas concentration is also improved.

In this case, the first gas sensor of the present invention may further include a reference-gas adjustment unit that causes the reference-gas adjustment pump cell to pump oxygen into the periphery of the pump reference electrode by applying a repeatedly turned ON/OFF control voltage to the reference-gas adjustment pump cell. The measurement pump cell controller may obtain the voltage of the measurement sensor cell in a period when the repeatedly turned ON/OFF control voltage is OFF, and may feedback-control the measurement pump cell so that the obtained voltage reaches the target voltage. As described above, the pump reference electrode and the voltage reference electrode are separately provided in the sensor element, thus at the time of pumping-in of oxygen performed by the reference-gas adjustment pump cell, a pump current does not flow through the voltage reference electrode. However, the control voltage applied to the reference-gas adjustment pump cell may affect the voltage of the measurement sensor cell. However, in the gas sensor, the voltage of the measurement sensor cell is obtained in an OFF period of the control voltage, thus the effect of the control voltage of the reference gas adjustment pump cell on the voltage of the measurement sensor cell can be reduced. Therefore, it is possible to prevent reduction in the accuracy of detection of the oxygen concentration due to the control voltage of the reference gas adjustment pump cell.

In this case, the measurement pump cell controller may obtain the voltage of the measurement sensor cell at the timing immediately before the subsequent turn-ON in an OFF-period of the control voltage which is repeatedly turned ON/OFF. In this manner, the effect of the control voltage on the voltage of the measurement sensor cell can be further prevented.

A second gas sensor of the present invention includes: the sensor element in which the aforementioned pump outer electrode and voltage outer electrode are separately provided; an adjustment chamber pump cell controller that causes the adjustment chamber pump cell to pump out oxygen from the oxygen concentration adjustment chamber or pump oxygen into the oxygen concentration adjustment chamber by controlling the adjustment chamber pump cell so that an oxygen concentration in the oxygen concentration adjustment chamber reaches a predetermined low concentration; and an oxygen concentration detector that detects an oxygen concentration in the measurement-object gas outside the element body based on the voltage of the outer sensor cell.

In the second gas sensor, the adjustment chamber pump cell controller controls the adjustment chamber pump cell so that the oxygen concentration in the oxygen concentration adjustment chamber reaches a predetermined low concentration. In this situation, for example, when the oxygen concentration in the measurement-object gas is switched between a high state in which the oxygen concentration is higher than a predetermined low concentration and a low state, the adjustment chamber pump cell controller switches the direction of oxygen moved by the adjustment chamber pump cell to the reverse direction. Thus, the direction of the pump current which flows through the adjustment chamber pump cell is switched to the reverse direction. Therefore, when one electrode serves as the pump outer electrode as well as the voltage outer electrode, the change in the voltage of the outer sensor cell also becomes slow due to the time required for current change when the direction of the pump current flowing through the adjustment chamber pump cell is switched to the reverse direction. In contrast, the gas sensor of the present invention is provided with the pump outer electrode and the voltage outer electrode separately, thus the voltage of the outer sensor cell is not affected by the time required for change in the pump current which flows through the adjustment chamber pump cell, and therefore, the change in the voltage of the outer sensor cell does not become slow. In other words, when the oxygen concentration in the measurement-object gas is switched between a high state in which the oxygen concentration is higher than a predetermined low concentration and a low state, the responsiveness of the voltage of the outer sensor cell is not likely to reduce.

### BRIEF DESCRIPTION OF THE DRAWINGS

FIG. 1 is a schematic cross-sectional view schematically illustrating an example of the configuration of a gas sensor 100 in a first embodiment.

FIG. 2 is a cross-sectional view of a pump reference electrode 42p and a voltage reference electrode 42s.

FIG. 3 is a block diagram illustrating an electrical connection relationship between a control device 95 and the cells of a sensor element 101.

FIG. 4 is an explanatory chart illustrating an example of temporal change in voltage Vp3.

FIG. 5 is an explanatory chart illustrating an example of temporal change in voltage Vref.

FIG. 6 shows graphs illustrating a relationship between elapsed time and NO output change rate in an endurance test.

FIG. 7 is a schematic cross-sectional view of a gas sensor 200 in a second embodiment.

FIG. 8 shows graphs illustrating the change in response time of voltage Vref before and after a continuous test in atmosphere.

FIG. 9 shows graphs illustrating the manner of temporal change in voltage Vref in Examples 2, 3 after a continuous test in atmosphere.

FIG. 10 is a cross-sectional view of a pump reference electrode 42p and a voltage reference electrode 42s according to a modification.

FIG. 11 is a cross-sectional view of a pump reference electrode 42p and a voltage reference electrode 42s according to a modification.

FIG. 12 is a schematic cross-sectional view of a gas sensor 300 according to a modification.

FIG. 13 is a schematic cross-sectional view schematically illustrating an example of a gas sensor 900 in a conventional example.

### DETAILED DESCRIPTION OF THE INVENTION

### First Embodiment

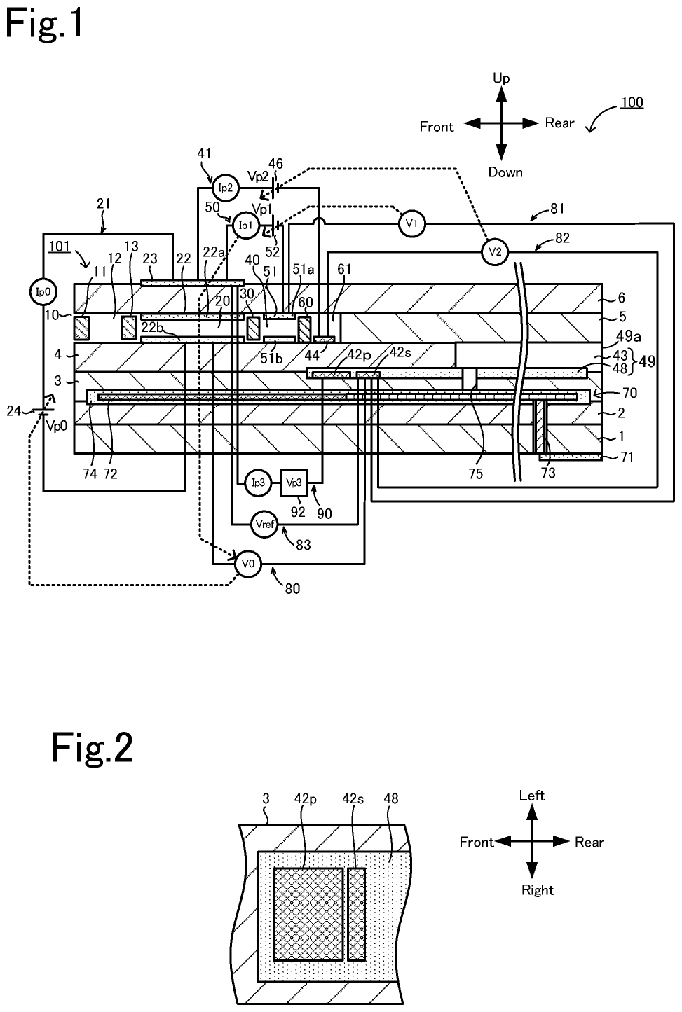

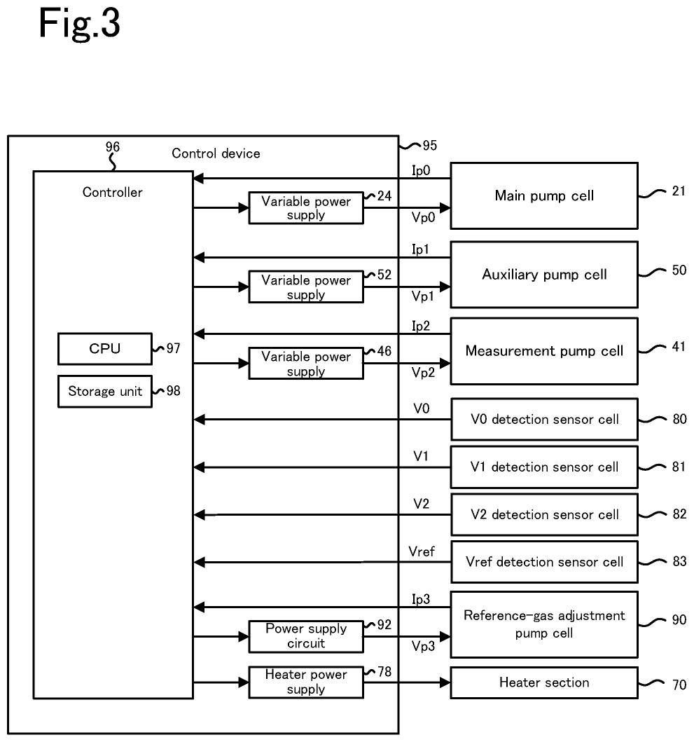

Next, an embodiment of the present invention will be described using drawings. FIG. 1 is a schematic cross-sectional view schematically illustrating an example of the configuration of a gas sensor 100 in a first embodiment of the present invention. FIG. 2 is a cross-sectional view of a pump reference electrode 42p and a voltage reference electrode 42s. FIG. 3 is a block diagram illustrating an electrical connection relationship between a control device 95 and the cells of the sensor element 101. The gas sensor 100 includes: the sensor element 101 having an elongate rectangular parallelepiped shape; and the control device 95 that controls the entire gas sensor 100. The gas sensor 100 also includes: an element sealing body (not illustrated) that seals and fixes the sensor element 101; and a bottomed cylindrical protective cover (not illustrated) that protects the front end of the sensor element 101. The sensor element 101 includes cells 21, 41, 50, 80 to 83, 90 and a heater section 70.

The gas sensor 100 is mounted on a pipe such as the exhaust gas pipe of an internal combustion engine, for example. The gas sensor 100 detects the concentration of a specific gas such as NOx and ammonia in a measurement-object gas which is an exhaust gas of an internal combustion engine. In this embodiment, the gas sensor 100 measures the NOx concentration as the specific gas concentration. The longitudinal direction (i.e., the left-right direction in FIG. 1) of the sensor element 101 is defined as the front-rear direction, and the thickness direction (i.e., the up-down direction in FIG. 1) of the sensor element 101 is defined as the up-down direction. Furthermore, the width direction (i.e., the direction perpendicular to the front-rear direction and the up-down direction) of the sensor element 101 is defined as the left-right direction. FIG. 2 illustrates a partial cross section around a pump reference electrode 42p and a voltage reference electrode 42s when a third substrate layer 3 is cut along the front-rear and left-right direction.

As illustrated in FIG. 1, the sensor element 101 has a layered body obtained by stacking six layers, namely, a first substrate layer 1, a second substrate layer 2, a third substrate layer 3, a first solid electrolyte layer 4, a spacer layer 5, and a second solid electrolyte layer 6 that are formed of oxygen-ion-conductive solid electrolyte layers composed of, for example, zirconia (ZrO₂), in that order from below in the drawing. The solid electrolyte used for forming each of these six layers is dense and hermetic. For example, the sensor element 101 is manufactured by performing predetermining processing and printing of a circuit pattern on ceramic green sheets corresponding to the individual layers, subsequently stacking the sheets, and then combining the sheets by calcination.

On the leading end side (front end side) of the sensor element 101 and between the lower surface of the second solid electrolyte layer 6 and the upper surface of the first solid electrolyte layer 4, a gas inlet 10, a first diffusion control section 11, a buffer space 12, a second diffusion control section 13, a first internal cavity 20, a third diffusion control section 30, a second internal cavity 40, a fourth diffusion control section 60, and a third internal cavity 61 are adjacently formed in that order to communicate with each other.

The gas inlet 10, the buffer space 12, the first internal cavity 20, the second internal cavity 40, the third internal cavity 61 form a space inside the sensor element 101, the space being provided by hollowing out the spacer layer 5 and partitioning the upper part of the space by the lower surface of the second solid electrolyte layer 6, the lower part by the upper surface of the first solid electrolyte layer 4, and the lateral part by the lateral surface of the spacer layer 5.

The first diffusion control section 11, the second diffusion control section 13, and the third diffusion control section 30 are each provided as two horizontally long slits (with an opening having a longitudinal direction in the direction perpendicular to the drawing). In addition, the fourth diffusion control section 60 is provided as one horizontally long slit (with an opening having a longitudinal direction in the direction perpendicular to the drawing) formed as a gap from the lower surface of the second solid electrolyte layer 6. Note that the portion from the gas inlet 10 to the third internal cavity 61 is also referred to as the measurement-object gas flow portion.

The sensor element 101 includes a reference-gas introduction portion 49 that causes a reference gas for measuring the NOx concentration to flow through a pump reference electrode 42p and a voltage reference electrode 42s from the outside of the sensor element 101. The reference-gas introduction portion 49 has a reference-gas introduction space 43, and a reference-gas introduction layer 48. The reference-gas introduction space 43 is a space provided inwardly from the rear end surface of the sensor element 101. The reference-gas introduction space 43 is provided between the upper surface of the third substrate layer 3 and the lower surface of the spacer layer 5, and at the position where the lateral part is partitioned by the lateral surface of the first solid electrolyte layer 4. The reference-gas introduction space 43 has an opening in the rear end surface of the sensor element 101, and a reference gas is introduced into the reference-gas introduction space 43 through the opening. The reference-gas introduction portion 49 guides the reference gas introduced from the outside of the sensor element 101 to the pump reference electrode 42p and the voltage reference electrode 42s, while adding a predetermined diffusion resistance to the reference gas. In this embodiment, the reference gas is an atmospheric gas.

The reference-gas introduction layer 48 is provided between the upper surface of the third substrate layer 3 and the lower surface of the first solid electrolyte layer 4. The reference-gas introduction layer 48 is a porous body composed of ceramics such as alumina. Part of the upper surface of the reference-gas introduction layer 48 is exposed to the reference-gas introduction space 43. The reference-gas introduction layer 48 is formed to cover the pump reference electrode 42p and the voltage reference electrode 42s. The reference-gas introduction layer 48 causes the reference gas to flow from the reference-gas introduction space 43 to the pump reference electrode 42p and the voltage reference electrode 42s. The reference-gas introduction portion 49 does not need to include the reference-gas introduction space 43. In that case, the reference-gas introduction layer 48 should be exposed to the rear end surface of the sensor element 101.

The pump reference electrode 42p and the voltage reference electrode 42s are interposed between the upper surface of the third substrate layer 3 and the first solid electrolyte layer 4, and as described above, in the periphery of the reference electrode 42, the reference-gas introduction layer 48 connected to the reference-gas introduction space 43 is provided. Furthermore, as will be described later, the voltage reference electrode 42s can be used to measure the oxygen concentration (oxygen partial pressure) in the first internal cavity 20, the second internal cavity 40, and the third internal cavity 61.

In the measurement-object gas flow portion, the gas inlet 10 is a section which is opened to the exterior space, and is designed to take the measurement-object gas into the sensor element 101 from the exterior space through the gas inlet 10. The first diffusion control section 11 adds a predetermined diffusion resistance to the measurement-object gas taken through the gas inlet 10. The buffer space 12 is provided to guide the measurement-object gas introduced from the first diffusion control section 11 to the second diffusion control section 13. The second diffusion control section 13 adds a predetermined diffusion resistance to the measurement-object gas introduced from the buffer space 12 into the first internal cavity 20. When the measurement-object gas is introduced from the outside of the sensor element 101 into the first internal cavity 20, the measurement-object gas suddenly taken into the sensor element 101 through the gas inlet 10 by a pressure variation (pulsation of the exhaust gas pressure when the measurement-object gas is exhaust gas of an automobile) of the measurement-object gas in the exterior space is not directly introduced into the first internal cavity 20, but is introduced into the first internal cavity 20 after the pressure variation in the measurement-object gas is cancelled through the first diffusion control section 11, the buffer space 12, and the second diffusion control section 13. Consequently, the pressure variation in the measurement-object gas introduced into the first internal cavity 20 is almost negligible. The first internal cavity 20 is provided as a space to adjust the oxygen partial pressure in the measurement-object gas introduced through the second diffusion control section 13. The oxygen partial pressure is adjusted by the main pump cell 21 operating.

The main pump cell 21 is an electrochemical pump cell including: an inner pump electrode 22 having a ceiling electrode portion 22a provided on substantially the entire lower surface of the second solid electrolyte layer 6 facing the first internal cavity 20; an outer pump electrode 23 provided to be exposed to the exterior space in an area corresponding to the ceiling electrode portion 22a on the upper surface of the second solid electrolyte layer 6; and the second solid electrolyte layer 6 interposed by these electrodes.

The inner pump electrode 22 is formed across the upper and lower solid electrolyte layers (the second solid electrolyte layer 6 and the first solid electrolyte layer 4) defining the first internal cavity 20, and the spacer layer 5 that provides a sidewall. Specifically, the ceiling electrode portion 22a is formed on the lower surface of the second solid electrolyte layer 6 providing the ceiling surface of the first internal cavity 20, a bottom electrode portion 22b is formed on the upper surface of the first solid electrolyte layer 4 providing the bottom surface, and a lateral electrode portion (not illustrated) is formed on the lateral wall surface (inner surface) of the spacer layer 5, forming both sidewalls of the first internal cavity 20 so as to connect the ceiling electrode portion 22a and the bottom electrode portion 22b, so that these electrodes are disposed in a structure of a tunnel form at the arrangement position of the lateral electrode portion.

The inner pump electrode 22 contains a noble metal (e.g., at least one of Pt, Rh, Pd, Ru and Ir) having catalytic activity. The inner pump electrode 22 also contains a noble metal (e.g., Au) having a catalytic activity inhibition ability to inhibit the catalytic activity for a specific gas of the noble metal having catalytic activity. Thus, the inner pump electrode 22 to be in contact with the measurement-object gas has a decreased reducing ability for a specific gas (in this case, NOx) component in the measurement-object gas. The inner pump electrode 22 is preferably composed of a cermet containing a noble metal and an oxide (in this case, ZrO₂) having oxygen ion conductivity. In addition, the inner pump electrode 22 is preferably a porous body. In this embodiment, the inner pump electrode 22 is a porous cermet electrode composed of Pt containing 1% of Au and ZrO₂.

As with the inner pump electrode 22, the outer pump electrode 23 contains a noble metal having catalytic activity. As with the inner pump electrode 22, the outer pump electrode 23 may be composed of a cermet. The outer pump electrode 23 is preferably a porous body. In this embodiment, the outer pump electrode 23 is a porous cermet electrode composed of Pt and ZrO₂.

In the main pump cell 21, oxygen in the first internal cavity 20 can be pumped out to the exterior space or oxygen in the exterior space can be pumped into the first internal cavity 20 by applying a desired voltage Vp0 across the inner pump electrode 22 and the outer pump electrode 23 to cause a pump current Ip0 to flow in a positive direction or a negative direction between the inner pump electrode 22 and the outer pump electrode 23.

Furthermore, in order to detect the oxygen concentration (oxygen partial pressure) in an atmosphere in the first internal cavity 20, an electrochemical sensor cell, that is, a V0 detection sensor cell 80 (also referred to as an oxygen partial pressure detection sensor cell for main pump control) is formed by the inner pump electrode 22, the second solid electrolyte layer 6, the spacer layer 5, the first solid electrolyte layer 4, the third substrate layer 3, and the voltage reference electrode 42s.

The oxygen concentration (oxygen partial pressure) in the first internal cavity 20 can be found by measuring the voltage V0 in the V0 detection sensor cell 80. Furthermore, the pump current Ip0 is controlled by feedback-controlling the voltage Vp0 of a variable power supply 24 so that the voltage V0 reaches a target value. Thus, the oxygen concentration in the first internal cavity 20 can be maintained at a predetermined constant value. The voltage V0 is a voltage across the inner pump electrode 22 and the voltage reference electrode 42s.

The third diffusion control section 30 adds a predetermined diffusion resistance to the measurement-object gas whose oxygen concentration (oxygen partial pressure) is controlled by the operation of the main pump cell 21 in the first internal cavity 20, and introduces the measurement-object gas to the second internal cavity 40.

After the oxygen concentration (oxygen partial pressure) is adjusted in advance in the first internal cavity 20, the second internal cavity 40 is provided as a space for further adjusting the oxygen partial pressure, by the auxiliary pump cell 50, of the measurement-object gas introduced through the third diffusion control section 30. Therefore, the oxygen concentration in the second internal cavity 40 can be maintained at a constant level with high accuracy, thus highly accurate measurement of NOx concentration is made possible in the gas sensor 100.

The auxiliary pump cell 50 is an auxiliary electrochemical pump cell including: an auxiliary pump electrode 51 having a ceiling electrode portion 51a provided on substantially the entire lower surface of the second solid electrolyte layer 6 facing the second internal cavity 40; the outer pump electrode 23 (an appropriate electrode outside the sensor element 101 suffices without being limited to the outer pump electrode 23); and the second solid electrolyte layer 6.

The auxiliary pump electrode 51 is disposed in the second internal cavity 40 in a structure of a tunnel form as in the inner pump electrode 22 provided in the aforementioned first internal cavity 20. Specifically, the ceiling electrode portion 51a is formed for the second solid electrolyte layer 6 that provides the ceiling surface of the second internal cavity 40, the bottom electrode portion 51b is formed for the first solid electrolyte layer 4 that provides the bottom surface of the second internal cavity 40, and a lateral electrode portion (not illustrated) that connects the ceiling electrode portion 51a and the bottom electrode portion 51b is formed in each of both wall surfaces of the spacer layer 5, which provide the lateral wall of the second internal cavity 40, thereby implementing a structure of a tunnel form. Note that as in the inner pump electrode 22, the auxiliary pump electrode 51 is also formed using a material having a decreased reducing ability for NOx component in the measurement-object gas.

In the auxiliary pump cell 50, oxygen in an atmosphere in the second internal cavity 40 can be pumped out to the exterior space or oxygen can be pumped from the exterior space into the second internal cavity 40 by applying a desired voltage Vp1 across the auxiliary pump electrode 51 and the outer pump electrode 23.

Furthermore, in order to control the oxygen partial pressure in an atmosphere in the second internal cavity 40, an electrochemical sensor cell, that is, a V1 detection sensor cell 81 (also referred to as an auxiliary-pump-control oxygen-partial-pressure detection sensor cell) is formed by the auxiliary pump electrode 51, the voltage reference electrode 42s, the second solid electrolyte layer 6, the spacer layer 5, the first solid electrolyte layer 4, and the third substrate layer 3.

Note that the auxiliary pump cell 50 performs pumping using a variable power supply 52 whose voltage is controlled based on the voltage V1 detected by the V1 detection sensor cell 81. Thus, the oxygen partial pressure in an atmosphere in the second internal cavity 40 is controlled at a low partial pressure which has substantially no effect on measurement of NOx. The voltage V1 is a voltage across the auxiliary pump electrode 51 and the voltage reference electrode 42s.

Along with this, the pump current Ip1 is used to control the electromotive force of the V0 detection sensor cell 80. Specifically, the pump current Ip1 is input to the V0 detection sensor cell 80 as a control signal, and the aforementioned target value of the voltage V0 is controlled so that the slope of the oxygen partial pressure in the measurement-object gas introduced from the third diffusion control section 30 into the second internal cavity 40 is controlled at a constant level all the time. When the gas sensor 100 is used as an NOx sensor, the oxygen concentration in the second internal cavity 40 is maintained at a constant value around approximately 0.001 ppm by the operation of the main pump cell 21 and the auxiliary pump cell 50.

The fourth diffusion control section 60 adds a predetermined diffusion resistance to the measurement-object gas whose oxygen concentration (oxygen partial pressure) is controlled by the operation of the auxiliary pump cell 50 in the second internal cavity 40, and introduces the measurement-object gas to the third internal cavity 61. The fourth diffusion control section 60 has a function of regulating the amount of NOx which flows into the third internal cavity 61.

After the oxygen concentration (oxygen partial pressure) is adjusted in advance in the second internal cavity 40, the third internal cavity 61 is provided as a space to perform a process related to measurement of the nitrogen oxide (NOx) concentration in the measurement-object gas on the measurement-object gas introduced through the fourth diffusion control section 60. The NOx concentration is mainly measured by the operation of the measurement pump cell 41 in the third internal cavity 61.

The measurement pump cell 41 measures the NOx concentration in the measurement-object gas in the third internal cavity 61. The measurement pump cell 41 is an electrochemical pump cell including: the measurement electrode 44 provided on the upper surface of the first solid electrolyte layer 4 facing the third internal cavity 61; the outer pump electrode 23; the second solid electrolyte layer 6; the spacer layer 5; and the first solid electrolyte layer 4. The measurement electrode 44 is a porous cermet electrode composed of a material which has a higher reducing ability for NOx component in the measurement-object gas than the reducing ability of the inner pump electrode 22. The measurement electrode 44 also functions as an NOx reduction catalyst to reduce the NOx present in an atmosphere in the third internal cavity 61.

In the measurement pump cell 41, the oxygen generated by decomposition of nitrogen oxide in an atmosphere in the periphery of the measurement electrode 44 is pumped out, and the amount of generated oxygen can be detected as the pump current Ip2.

In order to detect the oxygen partial pressure in the periphery of the measurement electrode 44, an electrochemical sensor cell, that is, a V2 detection sensor cell 82 (also referred to as a measurement-pump-control oxygen-partial-pressure detection sensor cell) is formed by the first solid electrolyte layer 4, the third substrate layer 3, the measurement electrode 44, and the voltage reference electrode 42s. A variable power supply 46 is controlled based on the voltage V2 detected by the V2 detection sensor cell 82. The voltage V2 is a voltage across the measurement electrode 44s and the voltage reference electrode 42s.

The measurement-object gas introduced into the second internal cavity 40 reaches the measurement electrode 44 in the third internal cavity 61 through the fourth diffusion control section 60 in a situation where the oxygen partial pressure is controlled. The nitrogen oxide in the measurement-object gas in the periphery of the measurement electrode 44 is reduced (2NO→N₂+O₂) to produce oxygen. The produced oxygen is then pumped by the measurement pump cell 41, and in this process, voltage Vp2 of the variable power supply 46 is controlled so that the voltage V2 detected by the V2 detection sensor cell 82 is constant (target value). The amount of oxygen produced in the periphery of the measurement electrode 44 is in proportion to the concentration of nitrogen oxide in the measurement-object gas, thus the nitrogen oxide concentration in the measurement-object gas is calculated using the pump current Ip2 in the measurement pump cell 41.

An electrochemical Vref detection sensor cell 83 is formed by the second solid electrolyte layer 6, the spacer layer 5, the first solid electrolyte layer 4, the third substrate layer 3, the outer pump electrode 23, and the voltage reference electrode 42s, and the oxygen partial pressure in the measurement-object gas outside the sensor is detectable with the voltage Vref obtained by the Vref detection sensor cell 83. The voltage Vref is a voltage across the outer pump electrode 23 and the voltage reference electrode 42s.

Furthermore, an electrochemical reference-gas adjustment pump cell 90 is formed by the second solid electrolyte layer 6, the spacer layer 5, the first solid electrolyte layer 4, the third substrate layer 3, the outer pump electrode 23, and the pump reference electrode 42p. The reference-gas adjustment pump cell 90 pumps oxygen by flowing the pump current Ip3 using a control voltage (voltage Vp3) applied by a power supply circuit 92 connected between the outer pump electrode 23 and the pump reference electrode 42p. Thus, the reference-gas adjustment pump cell 90 pumps oxygen from the space around the outer pump electrode 23 into the periphery of pump reference electrode 42p.

In the gas sensor 100 having such a configuration, the measurement-object gas having an oxygen partial pressure always maintained at a constant low value (a value having substantially no effect on measurement of NOx) is provided to the measurement pump cell 41 by operating the main pump cell 21 and the auxiliary pump cell 50. Therefore, the NOx concentration in the measurement-object gas can be found based on the pump current Ip2 which flows by pumping-out of oxygen by the measurement pump cell 41, the oxygen being produced by reduction of NOx in amount approximately proportional to the concentration of NOx in the measurement-object gas.

Furthermore, in order to enhance oxygen ion conductivity of the solid electrolyte, the sensor element 101 includes a heater section 70 having a role of temperature adjustment for heating the sensor element 101 and maintaining its temperature. The heater section 70 includes a heater connector electrode 71, a heater 72, a through-hole 73, a heater insulation layer 74, and a pressure diffusion hole 75.

The heater connector electrode 71 is formed to be in contact with the lower surface of the first substrate layer 1. Connecting the heater connector electrode 71 and an external power supply makes it possible to supply power to the heater section 70 from the outside.

The heater 72 is an electrical resistor which is formed to be interposed vertically between the second substrate layer 2 and the third substrate layer 3. The heater 72 is coupled to the heater connector electrode 71 via the through-hole 73, generates heat by being supplied with power from the outside through the heater connector electrode 71, and heats and maintains the temperature of the solid electrolyte forming the sensor element 101.

The heater 72 is buried over the entire region from the first internal cavity 20 to the third internal cavity 61, and the entire sensor element 101 can be adjusted to a temperature at which the solid electrolyte is activated.

The heater insulation layer 74 is composed of an insulator such as alumina on the upper and lower surfaces of the heater 72. The heater insulation layer 74 is formed for the purpose of obtaining an electrical insulating property between the second substrate layer 2 and the heater 72 as well as an electrical insulating property between the third substrate layer 3 and the heater 72.

The pressure diffusion hole 75 is a section provided to penetrate the third substrate layer 3 and the reference-gas introduction layer 48 so as to communicate with the reference-gas introduction space 43, and is formed for the purpose of reducing an internal pressure rise accompanied by a temperature increase in the heater insulation layer 74.

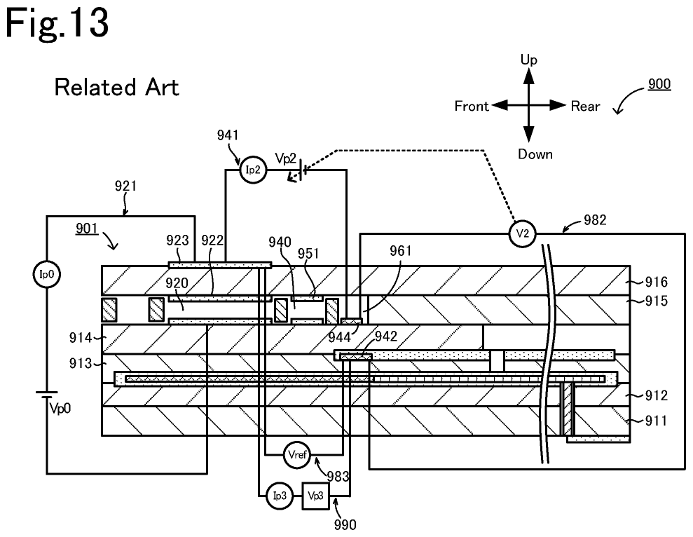

Here, the pump reference electrode 42p and the voltage reference electrode 42s will be described in detail. The pump reference electrode 42p and the voltage reference electrode 42s correspond to an aspect in which the reference electrode 942 in FIG. 13 is divided into two electrodes. Specifically, the reference electrode 942 in FIG. 13 serves as the electrode of the reference-gas adjustment pump cell 990 to cause the pump current Ip3 to flow, the electrode of the measurement-pump-control oxygen-partial-pressure detection sensor cell 982 to detect the voltage V2, and the electrode of the Vref detection sensor cell 983 to detect the voltage Vref. In contrast, in this embodiment, the pump reference electrode 42p of the reference-gas adjustment pump cell 90, and the voltage reference electrode 42s of the V0 detection sensor cell 80, the V1 detection sensor cell 81, the V2 detection sensor cell 82 and the Vref detection sensor cell 83 are both disposed as independent electrodes so as to be in contact with the reference gas introduced into the reference-gas introduction portion 49.

In this embodiment, as illustrated in FIG. 2, the pump reference electrode 42p and the voltage reference electrode 42s each have an approximately quadrangle shape in a top view. The voltage reference electrode 42s is located rearward of the pump reference electrode 42p. The voltage reference electrode 42s is shorter in length in the front-rear direction and smaller in area than the pump reference electrode 42p. Note that the area of an electrode is the one as seen in the direction perpendicular to the surface where the electrode is disposed. For example, the areas of the pump reference electrode 42p and the voltage reference electrode 42s are each an area in a top view.

The pump reference electrode 42p and the voltage reference electrode 42s may each contain a noble metal (e.g., at least one of Pt, Rh, Pd, Ru and Ir) having catalytic activity, or may be a conductive oxide sintered body containing a crystalline phase composed of a perovskite conductive oxide containing at least La, Fe and Ni. When the pump reference electrode 42p and the voltage reference electrode 42s contain a noble metal, the pump reference electrode 42p and the voltage reference electrode 42s are preferably composed of a cermet containing a noble metal and an oxide (in this case, ZrO₂) having oxygen ion conductivity. In addition, the pump reference electrode 42p and the voltage reference electrode 42s are preferably a porous body. The noble metal contained in the pump reference electrode 42p and the noble metal contained in the voltage reference electrode 42s may be the same in each of type and content ratio, or may be different in one of type and content ratio. In addition, the pump reference electrode 42p and the voltage reference electrode 42s are each a porous cermet electrode composed of Pt and ZrO₂.

As illustrated in FIG. 3, the control device 95 includes the aforementioned variable power supplies 24, 46, 52, a heater power supply 78, the aforementioned power supply circuit 92, and a controller 96. The controller 96 is a microprocessor including a CPU 97, a RAM which is not illustrated, and a storage unit 98. The storage unit 98 is, for example, a non-volatile memory such as a ROM, which is a device that stores various data. The controller 96 receives inputs of the voltages V0 to V2 and the voltage Vref of the sensor cells 80 to 83. The controller 96 receives inputs of the pump currents Ip0 to Ip3 which flow the respective pump cells 21, 50, 41, 90. The controller 96 controls the voltages Vp0 to Vp3 output by the variable power supplies 24, 46, 52 and the power supply circuit 92 by outputting a control signal to the variable power supplies 24, 46, 52 and the power supply circuit 92, thereby controlling the pump cells 21, 41, 50, 90. The controller 96 controls the electric power to be supplied to the heater 72 by the heater power supply 78 by outputting a control signal to the heater power supply 78, thereby adjusting the temperature of the sensor element 101. The storage unit 98 stores the target value V0*, V1*, V2*, Ip1* mentioned below.

The controller 96 feedback-controls the voltage Vp0 of the variable power supply 24 so that the voltage V0 reaches a target value V0* (in other words, so that the oxygen concentration in the first internal cavity 20 reaches a target concentration).

The controller 96 feedback-controls the voltage Vp1 of the variable power supply 52 so that the voltage V1 reaches a constant value (referred to as a target value V1*) (in other words, so that the oxygen concentration in the second internal cavity 40 reaches a predetermined low oxygen concentration which has substantially no effect on measurement of NOx). Along with this, the controller 96 sets (feedback-controls) the target value V0* of the voltage V0 based on the pump current Ip1 so that the pump current Ip1 caused to flow by the voltage Vp1 reaches a constant value (referred to as a target value Ip1*). Consequently, the slope of the oxygen partial pressure in the measurement-object gas introduced from the third diffusion control section 30 into the second internal cavity 40 becomes constant all the time. In addition, the oxygen partial pressure in an atmosphere in the second internal cavity 40 is controlled at a low partial pressure which has substantially no effect on measurement of NOx. The target value V0* is set to a value that causes the oxygen concentration in the first internal cavity 20 to be higher than 0% and reach a low oxygen concentration.

The controller 96 feedback-controls the voltage Vp2 of the variable power supply 46 so that the voltage V2 reaches a constant value (referred to as a target value V2*) (in other words, the oxygen concentration in the third internal cavity 61 reaches a predetermined low concentration). Thus, oxygen is pumped out from the third internal cavity 61 so that the oxygen produced by reducing the specific gas (in this case, NOx) in the measurement-object gas in the third internal cavity 61 becomes substantially zero. The controller 96 then obtains the pump current Ip2 as a detection value corresponding to the oxygen produced from NOx in the third internal cavity 61, and calculates the NOx concentration in the measurement-object gas based on the pump current Ip2. The target value V2* is a predetermined value such that the pump current Ip2 caused to flow by the feedback-controlled voltage Vp2 becomes a limiting current. The storage unit 98 stores a relational expression (e.g., the expression of a linear function) and a map as a correspondence relationship between the pump current Ip2 and the NOx concentration. Such a relational expression and a map can be determined by an experiment in advance. The controller 96 then detects the NOx concentration in the measurement-object gas based on the obtained pump current Ip2 and the aforementioned correspondence relationship stored in the storage unit 98. In this manner, oxygen from the specific gas in the measurement-object gas introduced into the sensor element 101 is pumped out, and the specific gas concentration is detected based on the amount of oxygen pumped out (based on the pump current Ip2 in this embodiment). This method is referred to as a limiting current method.

The controller 96 causes the pump current Ip3 to flow by controlling the power supply circuit 92 so that the voltage Vp3 is applied to the reference-gas adjustment pump cell 90. The flowing of the pump current Ip3 causes the reference-gas adjustment pump cell 90 to pump in oxygen from the periphery of the outer pump electrode 23 to the periphery of pump reference electrode 42p.

The function of the reference-gas adjustment pump cell 90 will be described below. The measurement-object gas which has flowed into the aforementioned protective cover (not illustrated) is introduced to a measurement-object gas flow portion, such as the gas inlet 10 of the sensor element 101. In contrast, a reference gas (atmosphere) is introduced to the reference-gas introduction portion 49 of the sensor element 101. The gas inlet 10 side of the sensor element 101 and the entry side of the reference-gas introduction portion 49, in short, the front end side and the rear end side of the sensor element 101 are partitioned and sealed by the aforementioned element sealing body (not illustrated) to prevent flow of gas between the sides. However, when the pressure on the side of measurement-object gas is high, the measurement-object gas may slightly enter the reference-gas side, and the oxygen concentration of the reference gas in the periphery of the rear end side of the sensor element 101 may decrease. At this point, if the oxygen concentration in the periphery of the voltage reference electrode 42s also decreases, the reference potential which is the electrical potential of the voltage reference electrode 42s also changes. The voltages V0 to V2, Vref of the sensor cells 80 to 83 mentioned above are each a voltage relative to the electrical potential of the voltage reference electrode 42s, thus when the reference potential changes, the accuracy of detection of the NOx concentration in the measurement-object gas may decrease. The reference-gas adjustment pump cell 90 serves to prevent such decrease in the detection accuracy. The control device 95 controls the power supply circuit 92, and applies, as the voltage Vp3, a pulse voltage repeatedly turned ON and OFF with a predetermined cycle (e.g., 10 msec) across the pump reference electrode 42p of the reference-gas adjustment pump cell 90 and the outer pump electrode 23. The flowing of the pump current Ip3 through the reference-gas adjustment pump cell 90 caused by the voltage Vp3 allows oxygen to be pumped into the periphery of the pump reference electrode 42p from the periphery of the outer pump electrode 23. Consequently, as described above, when the measurement-object gas causes the oxygen concentration to decrease in the periphery of the voltage reference electrode 42s, the decreased oxygen can be supplemented, and reduction in the accuracy of detection of the NOx concentration can be prevented. Note that the oxygen pumped into the periphery of the pump reference electrode 42p by the reference-gas adjustment pump cell 90 also reaches the periphery of the voltage reference electrode 42s through the reference-gas introduction layer 48. Thus, even when the pump reference electrode 42p and the voltage reference electrode 42s are separately provided in the reference-gas introduction portion 49, when the oxygen concentration in the periphery of the voltage reference electrode 42s decreases, the decreased oxygen can be supplemented by the reference-gas adjustment pump cell 90.

Note that in addition to the variable power supplies 24, 46, 52, the heater power supply 78 and the power supply circuit 92 which are illustrated in FIG. 3, the control device 95 is actually connected to the electrodes inside the sensor element 101 through unillustrated lead wires formed in the sensor element 101, and unillustrated connector electrodes (only the heater connector electrode 71 is illustrated in FIG. 1) formed on the rear end side of the sensor element 101.

The process performed by the controller 96 at the time of detection of the NOx concentration in the measurement-object gas by the gas sensor 100 will be described. First, the CPU 97 of the controller 96 starts to drive the sensor element 101. Specifically, the CPU 97 transmits a control signal to the heater power supply 78 to heat the sensor element 101 by the heater 72. The CPU 97 then heats the sensor element 101 to a predetermined driving temperature (e.g., 800° C.). Next, the CPU 97 starts to control the aforementioned pump cells 21, 41, 50, 90, and obtain the voltages V0 to V2, Vref from the aforementioned sensor cells 80 to 83. When the measurement-object gas is introduced through the gas inlet 10 in this state, the measurement-object gas passes through the first diffusion control section 11, the buffer space 12 and the second diffusion control section 13, and reaches the first internal cavity 20. Next, the oxygen concentration of the measurement-object gas is adjusted by the main pump cell 21 and the auxiliary pump cell 50 in the first internal cavity 20 and the second internal cavity 40, and the measurement-object gas after the adjustment reaches the third internal cavity 61. The CPU 97 then detects the NOx concentration in the measurement-object gas based on the obtained pump current Ip2 and the correspondence relationship stored in the storage unit 98.

As described above, the sensor element 101 of the gas sensor 100 includes the reference-gas adjustment pump cell 90 and the V2 detection sensor cell 82. The reference-gas adjustment pump cell 90 pumps oxygen into the periphery of the pump reference electrode 42p, thus reduction in the oxygen concentration of the reference gas in the reference-gas introduction portion 49 can be supplemented. In the V2 detection sensor cell 82, the voltage V2 based on the oxygen concentration difference between the reference gas and the third internal cavity 61 is generated, thus the oxygen concentration in the periphery of the measurement electrode 44 can be detected with the voltage V2 of the V2 detection sensor cell 82. In the sensor element 101, the pump reference electrode 42p and the voltage reference electrode 42s are separately provided as the electrodes to be in contact with the reference gas in the reference-gas introduction portion 49. Thus, unlike when one electrode serves as the pump reference electrode 42p as well as the voltage reference electrode 42s (e.g., in the sensor element 901 illustrated in FIG. 13, the reference electrode 942 serves as the electrode of the reference-gas adjustment pump cell 990 as well as the electrode of the measurement-pump-control oxygen-partial-pressure detection sensor cell 982), the pump current Ip3 at the time of pumping-in of oxygen performed by the reference-gas adjustment pump cell 90 does not flow through the voltage reference electrode 42s of the sensor element 101. Thus, the voltage V2 of the measurement pump cell 41 does not include a voltage drop of the voltage reference electrode 42s due to the pump current Ip3. Consequently, in the sensor element 101, it is possible to prevent reduction in the accuracy of detection of the oxygen concentration in the third internal cavity 61 due to the pump current Ip3 at the time of pumping-in of oxygen, while oxygen is being pumped into the reference-gas introduction portion 49. Therefore, in the sensor element 101, the voltage V2 has a value which corresponds with higher accuracy to the oxygen concentration in the third internal cavity 61, thus the accuracy of detection the oxygen concentration in the third internal cavity 61 using the V2 detection sensor cell 82 is improved. Based upon the foregoing, in the sensor element 101, it is possible to prevent reduction in the accuracy of detection of the oxygen concentration due to the pump current Ip3 at the time of pumping-in of oxygen, while oxygen is being pumped into the reference-gas introduction portion 49.

As described above, the voltage V2 is used to control the measurement pump cell 41, thus the NOx concentration in the measurement-object gas has more effect on the accuracy of detection of the oxygen concentration using the V2 detection sensor cell 82 than on the accuracy of detection of the oxygen concentration using the V0 detection sensor cell 80 or the V1 detection sensor cell 81. Thus, the accuracy of detection of the NOx concentration is improved by improving the accuracy of detection of the oxygen concentration in the third internal cavity 61 using the V2 detection sensor cell 82.

Note that when one reference electrode 942 is provided without having the pump reference electrode 42p and the voltage reference electrode 42s independently as in the sensor element 901 in a conventional example, in addition to the electromotive force based on the oxygen concentration difference between the periphery of the measurement electrode 944 and the periphery of the reference electrode 942, the voltage V2 of the measurement-pump-control oxygen-partial-pressure detection sensor cell 982 includes the value (voltage drop) obtained by multiplying the pump current Ip3 of the reference-gas adjustment pump cell 990 by the resistance of the reference electrode 942. Regarding the magnitude of a voltage drop in the reference electrode 942, due to the effect of a manufacturing variation (e.g., a variation in state, such as thickness, degree of porosity, surface area) of the reference electrode 942, when multiple sensor elements 901 are manufactured, individual difference may occur for each sensor element 901. Thus, in the sensor element 901, the accuracy of detection of the oxygen concentration in the third internal cavity 961 with the voltage V2 may have a variation for each sensor element 901. In contrast, in the sensor element 101 in this embodiment, a voltage drop does not occur in the voltage reference electrode 42s because no pump current Ip2 flows through the voltage reference electrode 42s, thus even when a plurality of sensor elements 101 have a manufacturing variation in the voltage reference electrode 42s, the accuracy of detection of the oxygen concentration in the third internal cavity 61 with the voltage V2 is unlikely to have a variation.

Note that in the sensor element 101, as with the voltage V2, the voltages V0, V1, Vref do not include a voltage drop of the voltage reference electrode 42s either due to the pump current Ip3. Therefore, the voltages V0, V1, Vref have values which correspond with high accuracy to the oxygen concentration in the first internal cavity 20, the oxygen concentration in the second internal cavity 40, and the oxygen concentration in the measurement-object gas outside the sensor element 101, respectively. In addition, even when a plurality of sensor elements 101 have a manufacturing variation in the voltage reference electrode 42s, the accuracy of detection of the oxygen concentration in each of the first internal cavity 20, the second internal cavity 40, and the outside of the sensor element 101 with the voltages V0, V1, Vref is unlikely to have a variation.

As described above, the controller 96 causes the measurement pump cell 41 to pump out oxygen from the third internal cavity 61 by feedback-controlling the measurement pump cell 41 so that the voltage V2 of the V2 detection sensor cell 82 reaches a target voltage (target value V2*). As described above, in the sensor element 101 in this embodiment, the accuracy of detection of the oxygen concentration in the third internal cavity 61 using the V2 detection sensor cell 82 has been improved, thus the oxygen concentration in the third internal cavity 61 can be adjusted with high accuracy to the oxygen concentration corresponding to the target value V2* by performing the aforementioned feedback control so that the voltage V2 reaches the target value V2*. In addition, the NOx concentration is detected based on the pump current Ip2 which flows through the measurement pump cell 41 by this feedback control, thus the accuracy of detection of the NOx concentration is also improved.

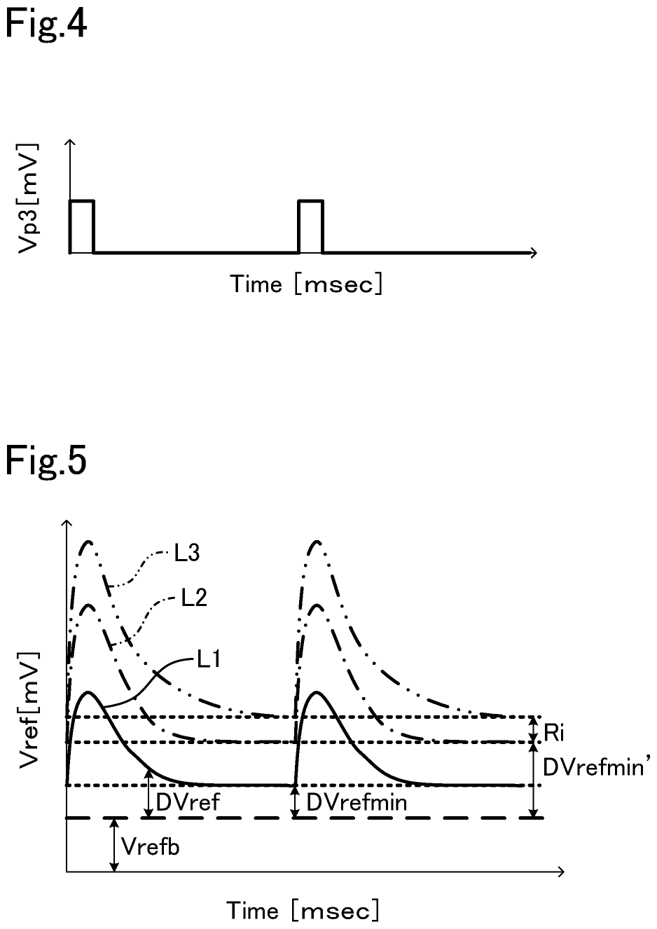

As described above, no pump current Ip3 flows through the voltage reference electrode 42s by separately providing the pump reference electrode 42p and the voltage reference electrode 42s, thus the voltage V2 does not include a voltage drop of the voltage reference electrode 42s. However, the voltage Vp3 applied to the reference-gas adjustment pump cell 90 may affect the voltage V2. This will be described. FIG. 4 is an explanatory chart illustrating an example of temporal change in the voltage Vp3. FIG. 5 is an explanatory chart illustrating an example of temporal change in the voltage Vref. When the pulse voltage in FIG. 4 is applied across the pump reference electrode 42p and the outer pump electrode 23 as the voltage Vp3, the voltage Vref across the voltage reference electrode 42s and the outer pump electrode 23 varies like the solid line waveform L1 in FIG. 5. Specifically, when the pulse voltage of the voltage Vp3 is turned ON, the voltage Vref gradually rises accordingly, while when the pulse voltage of the voltage Vp3 is turned OFF, the voltage Vref gradually falls accordingly, and the voltage Vref has a minimum value immediately before the pulse voltage is turned ON subsequently. The reason why the voltage Vref varies in this manner is that the voltage Vref includes a voltage drop caused by the pump current Ip3 that flows through the outer pump electrode 23. Specifically, rise and fall of the pump current Ip3 is repeated due to the pulse voltage as in the waveform L1 in FIG. 5, thus the magnitude of the voltage drop of the outer pump electrode 23 also varies, and the voltage Vref varies like the waveform L1 in FIG. 5. When the voltage Vref varies, the voltage V2 also varies for the following reasons. As is seen from FIG. 1, a relationship of |V2|=|Vp2|+|Vref| holds between the voltages Vref, Vp2, V2. Thus, when the voltage Vref varies due to the magnitude of the voltage drop of the outer pump electrode 23, the voltage V2 tends to increase as the voltage Vref increases even if the voltage Vp2 does not change. Therefore, when the voltage Vref varies cyclically as the waveform L1 in FIG. 5, the voltage V2 also varies cyclically as the waveform L1 in the same manner. Therefore, the controller 96 preferably obtains the voltage V2 at a timing with a variation in the voltage Vref as small as possible, caused by such a voltage drop of the outer pump electrode 23. In FIG. 5, the original value (the voltage based on the oxygen concentration difference between the periphery of the voltage reference electrode 42s and the periphery of the outer pump electrode 23) of the voltage Vref is shown as base voltage Vrefb. Residual voltage DVref that is the difference between the voltage Vref and the base voltage Vrefb includes a voltage drop of the outer pump electrode 23. The lower the residual voltage DVref, the smaller the voltage drop of the outer pump electrode 23 due to the pump current Ip3 which is caused to flow by the voltage Vp3, thus the change in the voltage V2 due to the voltage drop is also small. Thus, the controller 96 preferably obtains the voltage V2 in a period when the voltage Vp3 is OFF, and more preferably, obtains the voltage V2 at a timing with a residual voltage DVref as low as possible in the OFF-period of the voltage Vp3. In this manner, the effect of the voltage Vp3 on the voltage V2 can be reduced. Therefore, reduction in the accuracy of measurement of the oxygen concentration in the third internal cavity 61, caused by the voltage Vp3 can be prevented, and the voltage V2 has a value which corresponds with higher accuracy to the oxygen concentration in the third internal cavity 61. In addition, when the controller 96 feedback-controls the measurement pump cell 41 based on the voltage V2 obtained at such timing, the oxygen concentration in the third internal cavity 61 can be adjusted with high accuracy to the oxygen concentration corresponding to the target value V2*.

Specifically, the timing with a residual voltage DVref as low as possible may be any timing in the following period. Specifically, first, in one cycle of ON and OFF of the voltage Vp3, the maximum of the value of the voltage Vref is assumed to be 100%, and the minimum is assumed to be 0%. Let the period with a low residual voltage DVref be the period since the voltage Vref falls below 10% after turn-OFF of the voltage Vp3 until the voltage Vref starts to rise due to turn-ON of the voltage Vp3 in the next cycle. The controller 96 preferably obtains the voltage V2 at any timing in this period. More preferably, the controller 96 obtains the voltage V2 at the timing of a minimum DVrefmin (see FIG. 5) of the residual voltage DVref in one cycle of ON and OFF of the voltage Vp3. When the voltage Vref is stable in an OFF-period of the voltage Vp3 (until the voltage Vp3 is turned ON subsequently) as in the waveform L1 in FIG. 5, the controller 96 may obtain the voltage V2 at any timing in the period in which the voltage Vref is stable. In this manner, the controller 96 can obtain the voltage V2 at the timing when the residual voltage DVref attains the minimum DVrefmin. In contrast, when the voltage Vref is unstable in an OFF-period of the voltage Vp3, the residual voltage DVref attains the minimum DVrefmin at the timing immediately before the subsequent turn-ON in the OFF-period of the voltage Vp3, thus the controller 96 preferably obtains the voltage V2 at this timing. The timing when the controller 96 obtains the voltage V2 can be determined in advance by an experiment based on the ON/OFF cycle of the voltage Vp3, and the waveforms of temporal change in the pump current Ip3 and the voltage Vref caused by the voltage Vp3.

Note that for the sake of explanation, FIG. 5 illustrates the waveform of the voltage Vref when the base voltage Vrefb is constant, specifically, when the oxygen concentration in the measurement-object gas in the periphery of the outer pump electrode 23 is constant. Actually, the base voltage Vrefb varies according to the oxygen concentration in the measurement-object gas in the periphery of the outer pump electrode 23, thus the voltage Vref also changes due to the variation in the base voltage Vrefb.

As with the voltage V2, the voltages V0, V1 are affected by the voltage Vp3. In addition, the voltage Vref is also affected by the voltage Vp3 as illustrated in FIG. 5. Thus, as with the voltage V2, the controller 96 obtains the voltages V0, V1, Vref preferably in an OFF-period of the voltage Vp3, more preferably in the aforementioned period with a low residual voltage DVref, and still more preferably at any timing in the period in which the voltage Vref is stable or at the timing immediately before the subsequent turn-ON in an OFF-period of the voltage Vp3. In addition, as with the voltage V2, the controller 96 obtains the pump currents Ip0 to Ip3 preferably in an OFF-period of the voltage Vp3, more preferably in the aforementioned period with a low residual voltage DVref, and still more preferably at any timing in the period in which the voltage Vref is stable or at the timing immediately before the subsequent turn-ON in an OFF-period of the voltage Vp3. In this embodiment, the controller 96 obtains the voltages V0, V1, V2, Vref, and the pump currents Ip0 to Ip3 at the timing immediately before the subsequent turn-ON in an OFF-period of the voltage Vp3.

Note that in the sensor element 101 in this embodiment, as described above, the residual voltage DVref includes the voltage drop of the outer pump electrode 23, but does not include the voltage drop of the voltage reference electrode 42s because the pump current Ip3 does not flow. In contrast, when one reference electrode 942 serves as the pump reference electrode 42p as well as the voltage reference electrode 42s as in the sensor element 901 illustrated in FIG. 13, the residual voltage DVref includes not only the voltage drop of the outer pump electrode 923, but also the voltage drop of the reference electrode 942. Thus, the voltage Vref of the sensor element 901 varies like the dashed line waveform L2 in FIG. 5. In the waveform L2, the residual voltage DVref has always a greater value than in the waveform L1, and accordingly, the voltage Vref is also higher than the waveform L1. Therefore, the minimum DVrefmin′ of the residual voltage DVref of the waveform L2 is also higher than the minimum DVrefmin of the waveform L1. Thus, in the sensor element 101 in this embodiment, the residual voltage DVref and the minimum DVrefmin can be reduced by separately disposing the pump reference electrode 42p and the voltage reference electrode 42s. Therefore, in the sensor element 101, the effect of the voltage Vp3 on the voltage V2 can be reduced than in the sensor element 901, thus the voltage V2 has a value which corresponds with higher accuracy to the oxygen concentration in the third internal cavity 61.

Disposing the pump reference electrode 42p and the voltage reference electrode 42s separately can prevent reduction (hereinafter referred to as “deterioration of the accuracy of detection”) in the accuracy of detection of the NOx concentration with use of the gas sensor 100. The reason for this will be described. When one reference electrode 942 serves as the pump reference electrode 42p as well as the voltage reference electrode 42s as in the sensor element 901 illustrated in FIG. 13, the noble metal in the reference electrode 942 may be oxidized by flowing the pump current Ip3. For example, when the reference electrode 942 contains Pt, part of the Pt may be oxidized to produce PtO, PtO₂. Oxidized noble metal is more likely to be evaporated than the noble metal before being oxidized, thus the noble metal in the reference electrode 942 decreases with use of the gas sensor 900, and the catalytic activity of the reference electrode 942 is reduced. In short, the reference electrode 942 deteriorates. When the catalytic activity of the reference electrode 942 is reduced, the reaction resistance of the reference electrode 942 increases and the voltage drop further increases, thus the residual voltage DVref of the waveform L2 in FIG. 5 further rises with use of the gas sensor 900. As a result, for example, as in the dashed-two dotted line waveform L3 illustrated in FIG. 5, the residual voltage DVref overall becomes higher than in the waveform L2 by rise Ri of the voltage drop of the reference electrode 942. In this manner, in the gas sensor 900, the residual voltage DVref at the time immediately after manufacturing is higher than in the gas sensor 100, and with use of the gas sensor 900, the residual voltage DVref further increases. Therefore, the effect of the voltage Vp3 on the voltage V2 also increases with use of the gas sensor 900, thus the accuracy of measurement of the oxygen concentration in the third internal cavity 61 by the V2 detection sensor cell 82 is reduced. Consequently, in the gas sensor 900, the accuracy of detection of the NOx concentration deteriorates. In contrast, in the gas sensor 100, the pump current Ip3 is not passed through the voltage reference electrode 42s, thus the voltage reference electrode 42s is unlikely to deteriorate. Even if the voltage reference electrode 42s deteriorates, the pump current Ip3 is not passed therethrough, thus a voltage drop does not occur. Because of this, even when the gas sensor 100 is used for a long time, the residual voltage DVref is unlikely to rise, thus the accuracy of detection of the oxygen concentration in the third internal cavity 61 with the voltage V2 is unlikely to decrease, and deterioration of the accuracy of detection of the NOx concentration is prevented. Note that also in the gas sensor 100, the residual voltage DVref may rise due to deterioration of the outer pump electrode 23. However, the outer pump electrode 23 has a relatively larger area than other electrodes, and the outer pump electrode 23 (and the inner pump electrode 22) are heated by the heater 72 to a relatively higher temperature than that of other electrodes, thus the resistance value of the outer pump electrode 23 is often lower than that of other electrodes. Thus, for example, as compared to the reference electrode 942 of the gas sensor 900, the amount of rise caused by the voltage drop due to deterioration of the outer pump electrode 23, in other words, the amount of rise of the residual voltage DVref is small.

Note that in addition to the aforementioned electromotive force based on the oxygen concentration difference between the periphery of the measurement electrode 44 and the periphery of the voltage reference electrode 42s, and the voltage drop of the outer pump electrode 23, the voltage V2 also includes the thermal electromotive force of the voltage reference electrode 42s. Thus, in order to further improve the accuracy of detection of the oxygen concentration using the V2 detection sensor cell 82, it is preferable to reduce the thermal electromotive force of the voltage reference electrode 42s. For example, a temperature variation in the voltage reference electrode 42s can be reduced by decreasing the area of the voltage reference electrode 42s as much as possible, thus the thermal electromotive force of the voltage reference electrode 42s can be reduced. The voltage reference electrode 42s may have a high resistance value because the pump current Ip3 does not flow therethrough, thus is more easily reduced in area than the pump reference electrode 42p. In this embodiment, as described above, the area of the voltage reference electrode 42s is made smaller than the area of the pump reference electrode 42p, thus the thermal electromotive force of the voltage reference electrode 42s can be made relatively small.

The pump reference electrode 42p and the voltage reference electrode 42s are preferably disposed as close as possible in a range where both are not in contact with each other (not conductive to each other). In this manner, the oxygen pumped into the periphery of the pump reference electrode 42p is likely to reach the periphery of the voltage reference electrode 42s, thus when the oxygen concentration in the periphery of the voltage reference electrode 42s decreases, the decreased oxygen can be supplemented by the reference-gas adjustment pump cell 90. In this embodiment, as illustrated in FIG. 2, the pump reference electrode 42p and the voltage reference electrode 42s are adjacent in the front-rear direction so that both are disposed as close as possible.

The manner of the aforementioned change in the accuracy of detection of the NOx concentration with use of the gas sensor has been studied in the following way. First, Example 1 is implemented by producing the sensor element 101 and the gas sensor 100 in this embodiment illustrated in FIGS. 1 to 3. In addition, Comparative Example 1 is implemented by producing a gas sensor which is the same as Example 1 except that the pump reference electrode 42p and the voltage reference electrode 42s are not included but the reference electrode 942 of FIG. 13 is included instead. In Comparative Example 1, the reference electrode 942 constitutes part of each of the reference-gas adjustment pump cell 90, the V0 detection sensor cell 80, the V1 detection sensor cell 81, the V2 detection sensor cell 82, and the Vref detection sensor cell 83. The same material is used for the pump reference electrode 42p and the voltage reference electrode 42s in Example 1, and the reference electrode 942 of Comparative Example 1.

An endurance test using a diesel engine was conducted for Example 1 and Comparative Example 1 to evaluate the degree of deterioration of the accuracy of detection of the NOx concentration. First, the gas sensor in Example 1 was mounted on a model gas device. The heater 72 was energized to attain a temperature of 800° C. to heat the sensor element 101. A state is achieved in which the aforementioned pump cells 21, 41, 50, 90 are controlled by the controller 96, and the voltages V0, V1, V2, Vref are obtained from the aforementioned sensor cells 80 to 83. In this state, a first model gas having a base gas of nitrogen and an NO concentration of 1500 ppm is passed through the model gas device, and the standby state is maintained until the pump current Ip2 is stabilized. The pump current Ip2 after stabilized was measured as an initial value Ia of the output of the gas sensor for NO. Subsequently, an endurance test was conducted as follows. First, the gas sensor in Example 1 was mounted on the exhaust gas pipe of an automobile. Then, a 40-minute operation pattern formed by an engine rotation speed in a range of 1500 to 3500 rpm and a load torque in a range of 0 to 350 N-m was repeated until 1000 hours have elapsed. Note that the gas temperature then was 200° C. to 600° C., and the NOx concentration was 0 to 1500 ppm. The controller 96 continued to control the aforementioned pump cells and obtain the voltages also during the 1000 hours. After lapse of 1000 hours, the gas sensor is temporarily removed from the exhaust gas pipe and is mounted on the model gas device, and the value of the pump current Ip2 was measured by the same method as for the initial value Ia to obtain value Ib after lapse of 1000 hours. NO output change rate [%] of the pump current Ip2 of the gas sensor in Example 1 after lapse of 1000 hours was derived from NO output change rate after lapse of 1000 hours=[1−(Ib/Ia)]×100%. Similarly, 1000-hour endurance test and subsequent measurement of the value Ib were repeatedly conducted, and NO output change rate was derived for the total elapsed time of the endurance test of each of 2000 hours and 3000 hours. For the gas sensor of Comparative Example 1, similarly, NO output change rate was derived for the initial value Ia and the elapsed time of the endurance test up to 3000 hours.

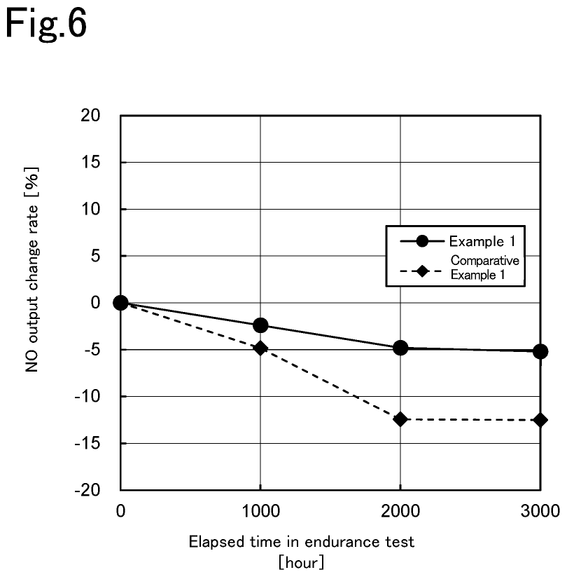

FIG. 6 shows graphs illustrating a relationship between elapsed time and NO output change rate in the aforementioned endurance test in Example 1 and Comparative Example 1. In each of Example 1 and Comparative Example 1, NO output change rate is shown, where the initial value Ia for the elapsed time of 0 hour is used as a reference (=NO output change rate is 0%). The smaller the absolute value of NO output change rate, the lower the change in the pump current Ip2 for NO after an endurance test, which shows that deterioration of the accuracy of detection of the NOx concentration is prevented. As illustrated in FIG. 6, as compared to Comparative Example 1 in which the reference electrode 942 is disposed instead of these electrodes, deterioration of the accuracy of detection of the NOx concentration is further prevented in Example 1 in which the pump reference electrode 42p and the voltage reference electrode 42s are both disposed. This is probably because whereas the reference electrode 942 of Comparative Example 1 deteriorates through an endurance test as described above, the pump reference electrode 42p in Example 1 is unlikely to deteriorate and even if the pump reference electrode 42p deteriorates, no voltage drop occurs due to the pump current Ip3.

The correspondence relationships between the components in this embodiment and the components in the present invention will now be clarified. The first substrate layer 1, the second substrate layer 2, the third substrate layer 3, the first solid electrolyte layer 4, the spacer layer 5 and the second solid electrolyte layer 6 correspond to an element body according to the present invention, the reference-gas introduction portion 49 corresponds to a reference-gas introduction portion, the pump reference electrode 42p corresponds to a pump reference electrode, the reference-gas adjustment pump cell 90 corresponds to a reference-gas adjustment pump cell, the voltage reference electrode 42s corresponds to a voltage reference electrode, the inner pump electrode 22, the auxiliary pump electrode 51, the measurement electrode 44 and the outer pump electrode 23 correspond to a measurement-object gas-side electrode, and the V0 detection sensor cell 80, the V1 detection sensor cell 81, the V2 detection sensor cell 82 and the Vref detection sensor cell 83 correspond to a sensor cell. In addition, the outer pump electrode 23 corresponds to a pumping-in source electrode. The third internal cavity 61 corresponds to a measurement chamber, the measurement pump cell 41 corresponds to a measurement pump cell, the measurement electrode 44 corresponds to a measurement electrode, and the V2 detection sensor cell 82 corresponds to a measurement sensor cell. The controller 96 corresponds to a measurement pump cell controller and a reference-gas adjustment unit.

In the gas sensor 100 in this embodiment described in detail above, the pump reference electrode 42p and the voltage reference electrode 42s are separately provided in the sensor element 101 as the electrodes to be in contact with the reference gas in the reference-gas introduction portion 49. Therefore, the voltages V0, V1, V2, Vref do not include a voltage drop of the voltage reference electrode 42s due to the pump current Ip3. Thus, the accuracy of detection of the oxygen concentration in the first internal cavity 20, the second internal cavity 40, the third internal cavity 61, and the outside of the sensor element 101 using the respective sensor cells 80 to 83 is improved. Based upon the foregoing, in the sensor element 101, it is possible to prevent reduction in the accuracy of detection of the oxygen concentration due to the pump current Ip3 at the time of pumping-in of oxygen, while oxygen is being pumped into the reference-gas introduction portion 49. Particularly, use of the voltage V2 of the V2 detection sensor cell 82 for the control of the measurement pump cell 41 gives more effects on the accuracy of detection of the specific gas concentration in the measurement-object gas than the voltages V0, V1, Vref. Thus, since the voltage reference electrode 42s provided independently from the pump reference electrode 42p constitutes part of the V2 detection sensor cell 82, the accuracy of detection of the specific gas concentration is further improved.

Furthermore, the controller 96 causes the measurement pump cell 41 to pump out oxygen from the third internal cavity 61 by feedback-controlling the measurement pump cell 41 so that the voltage V2 reaches the target value V2*. As described above, the accuracy of detection of the oxygen concentration in the third internal cavity 61 using the V2 detection sensor cell 82 of the sensor element 101 is improved by separately disposing the pump reference electrode 42p and the voltage reference electrode 42s, thus the oxygen concentration in the third internal cavity 61 can be adjusted with high accuracy to the oxygen concentration corresponding to the target value V2* by performing the aforementioned feedback control. In addition, the NOx concentration is detected by this feedback control based on the pump current Ip2 which flows through the measurement pump cell 41, thus the accuracy of detection of the NOx concentration is also improved.

Furthermore, the controller 96 applies the voltage Vp3 repeatedly turned ON and OFF to the reference-gas adjustment pump cell 90, thereby causing the reference-gas adjustment pump cell 90 to pump oxygen into the periphery of the pump reference electrode 42p. The controller 96 then obtains the voltage V2 of the V2 detection sensor cell 82 in an OFF-period of the voltage Vp3. Thus, the effect of the voltage Vp3 on the voltage V2 of the V2 detection sensor cell 82 can be reduced. Therefore, reduction in the accuracy of detection of the oxygen concentration due to the voltage Vp3 can be prevented.

### Second Embodiment

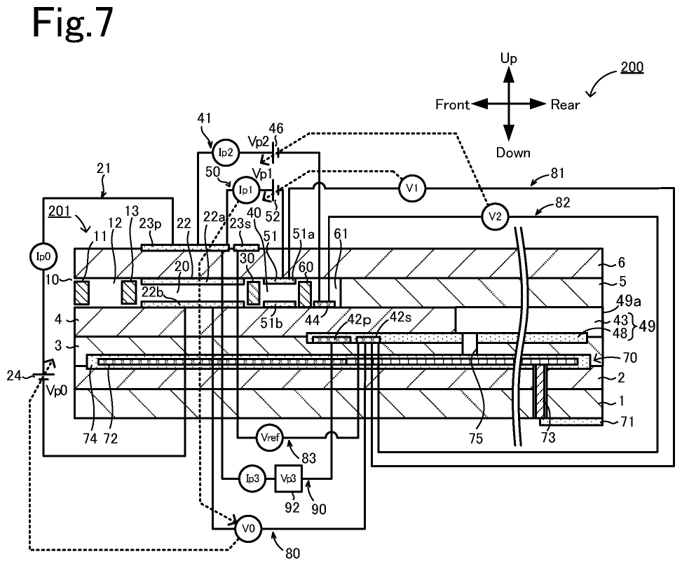

FIG. 7 is a schematic cross-sectional view schematically illustrating an example of a gas sensor 200 in a second embodiment. As with the sensor element 101, a sensor element 201 of the gas sensor 200 includes the pump reference electrode 42p and the voltage reference electrode 42s, and further includes a pump outer electrode 23p and a voltage outer electrode 23s instead of the outer pump electrode 23 in FIG. 1. The pump outer electrode 23p and the voltage outer electrode 23s are each disposed outside the sensor element 201 so as to be in contact with the measurement-object gas outside the sensor element 201. In this embodiment, as with the outer pump electrode 23, the pump outer electrode 23p and the voltage outer electrode 23s are disposed on the upper surface of the sensor element 201. The pump outer electrode 23p constitutes part of each of the main pump cell 21, the auxiliary pump cell 50, the measurement pump cell 41, and the reference-gas adjustment pump cell 90, thus the pump currents Ip0, Ip1, Ip2, Ip3 flow through the pump outer electrode 23p. The voltage outer electrode 23s constitutes part of the Vref detection sensor cell 83. Thus, the voltage across the voltage outer electrode 23s and the voltage reference electrode 42s is the voltage Vref. As with the pump reference electrode 42p and the voltage reference electrode 42s illustrated in FIG. 2, the pump outer electrode 23p and the voltage outer electrode 23s each have an approximately quadrangle shape in a top view. The voltage outer electrode 23s is located rearward of the pump outer electrode 23p. The voltage outer electrode 23s is shorter in length in the front-rear direction and smaller in area than the pump outer electrode 23p. The material for the pump outer electrode 23p and the voltage outer electrode 23s is the same as for the outer pump electrode 23 in the first embodiment. However, the noble metal contained in the pump outer electrode 23p and the noble metal contained in the voltage outer electrode 23s may be different in at least one of type and content ratio.

Except this point, the gas sensor 200 is the same as the gas sensor 100 in the first embodiment. For example, as in the first embodiment, the controller 96 feedback-controls the voltage Vp0 of the variable power supply 24 so that the voltage V0 reaches the target value V0*, thus the pump current Ip0 flows through the main pump cell 21. The controller 96 detects the oxygen concentration in the measurement-object gas outside the sensor element 201 based on the voltage Vref of the Vref detection sensor cell 83.

In the sensor element 201 of the gas sensor 200, as described above, the pump outer electrode 23p that constitutes part of each of the pump cells 21, 41, 50, 90, and the voltage outer electrode 23s that constitutes part of each of the Vref detection sensor cell 83 are both disposed outside the sensor element 201. In short, in the sensor element 201, the pump outer electrode 23p and the voltage outer electrode 23s are both disposed outside the sensor element 201. Thus, the same effect as the one achieved by separately providing the pump reference electrode 42p and the voltage reference electrode 42s in the above-described first embodiment is obtained. For example, unlike when one outer pump electrode 923 serves as the electrode of the measurement pump cell 941 as well as the electrode of the Vref detection sensor cell 983 as in the gas sensor 900 illustrated FIG. 13, the pump current Ip2 does not flow through the voltage outer electrode 23s. Similarly, the pump currents Ip0, Ip1, Ip3 do not flow through the voltage outer electrode 23s either. Thus, the voltage Vref of the Vref detection sensor cell 83 does not include a voltage drop of the voltage outer electrode 23s due to the pump currents Ip0 to Ip3. Consequently, the voltage Vref of the Vref detection sensor cell 83 has a value which corresponds with higher accuracy to the oxygen concentration in the measurement-object gas outside the sensor element 201, thus the accuracy of detection of the oxygen concentration in the measurement-object gas using the Vref detection sensor cell 83 is improved. In addition, even when a plurality of sensor elements 201 have a manufacturing variation in the voltage outer electrode 23s, the accuracy of detection of the oxygen concentration in the measurement-object gas outside the sensor element 201 with the voltage Vref is unlikely to have a variation.

The voltage Vref in the sensor element 201 is the voltage across the voltage outer electrode 23s and the voltage reference electrode 42s, and in the gas sensor 200, no pump current flows through each of the voltage outer electrode 23s and the voltage reference electrode 42s which are electrodes on both ends for measurement of the voltage Vref. Thus, in the sensor element 201, particularly, the voltage Vref has a value which corresponds to the oxygen concentration with higher accuracy than the voltages V0, V1, V2. In addition, the voltage Vref of the sensor element 201 has a value which corresponds to the oxygen concentration outside the sensor element with even higher accuracy than the voltage Vref of the sensor element 101.

As described above, the controller 96 controls the main pump cell 21 so that the voltage V0 reaches the target value V0*, in other words, the oxygen concentration in the first internal cavity 20 reaches a predetermined low concentration. In this situation, for example, when the oxygen concentration in the measurement-object gas is switched between a high state in which the oxygen concentration is higher than a predetermined low concentration and a low state, the controller 96 switches the direction of oxygen moved by the main pump cell 21 to the reverse direction. Thus, the direction of the pump current Ip0 which flows through the main pump cell 21 is switched to the reverse direction. For example, when the measurement-object gas is switched from a lean atmosphere to a rich atmosphere, the direction of the pump current Ip0 which flows through the main pump cell 21 is switched from the direction in which oxygen is pumped out from the first internal cavity 20 to the direction in which oxygen is pumped into the first internal cavity 20. The lean atmosphere indicates a state where the air-fuel ratio of the measurement-object gas is higher than a theoretical air-fuel ratio, and the rich atmosphere indicates a state where the air-fuel ratio of the measurement-object gas is lower than a theoretical air-fuel ratio. In a rich atmosphere, the measurement-object gas contains an unburnt fuel, and the right amount of oxygen required for burning the unburnt fuel corresponds to the oxygen concentration in the measurement-object gas in a rich atmosphere. Therefore, the oxygen concentration in the measurement-object gas in a rich atmosphere is expressed as a negative value. Thus, when the measurement-object gas is in a rich atmosphere, in order to change a negative oxygen concentration to a predetermined low concentration (a state where the oxygen concentration is higher than 0%) corresponding to the target value V0*, the controller 96 controls the main pump cell 21 to pump oxygen into the first internal cavity 20. Thus, when one electrode serves as the pump outer electrode 23p as well as the voltage outer electrode 23s, the change in the voltage Vref also becomes slow due to the time required for current change when the direction of the pump current Ip0 flowing through the main pump cell 21 is switched to the reverse direction. In contrast, in this embodiment, the pump outer electrode 23p and the voltage outer electrode 23s are separately provided, thus the voltage Vref is not affected by the time required for change in the pump current Ip0, and therefore, the change in the voltage Vref does not become slow. In other words, when the oxygen concentration in the measurement-object gas is switched between a high state in which the oxygen concentration is higher than a predetermined low concentration and a low state, the responsiveness of the voltage Vref is not likely to reduce.

In addition, when one electrode serves as the pump outer electrode 23p as well as the voltage outer electrode 23s, the electrode deteriorates with use, thus the aforementioned time required for current change when the direction of the pump current Ip0 is switched to the reverse direction may be further increased. This is probably because capacity components of the electrode change due to deterioration of the electrode. Thus, for example, in the gas sensor 900, the responsiveness of the voltage Vref may reduce with use (hereinafter referred to as “deterioration of responsiveness”). In contrast, in this embodiment, the voltage outer electrode 23s is unlikely to deteriorate because the pump currents Ip0 to Ip3 are not passed through the voltage outer electrode 23s. Even if the voltage outer electrode 23s deteriorates, the pump current Ip0 is not passed through the voltage outer electrode 23s, thus the voltage outer electrode 23s is not affected by switching of the direction of the pump current Ip0 to the reverse direction. Consequently, even when the sensor element 201 is used for a long time, the responsiveness of the voltage Vref is unlikely to deteriorate.

The responsiveness of the voltage Vref and the manner of deterioration of the responsiveness have been studied in the following way. First, Example 2 is implemented by producing the sensor element 201 and the gas sensor 200 in this embodiment illustrated in FIG. 7. In addition, Example 3 is implemented by producing a gas sensor which is the same as Example 2 except that the pump outer electrode 23p and the voltage outer electrode 23s are not included but the outer pump electrode 923 of FIG. 13 is included. In Example 3, the outer pump electrode 923 constitutes part of each of the main pump cell 21, the auxiliary pump cell 50, the measurement pump cell 41, the reference-gas adjustment pump cell 90, and the Vref detection sensor cell 83. The same material is used for the pump outer electrode 23p, and the voltage outer electrode 23s in Example 2, and the outer pump electrode 923 in Example 3.

For Examples 2, 3, the responsiveness of the voltage Vref was studied. First, the gas sensor in Example 2 was mounted on a pipe. The heater 72 was energized to attain a temperature of 800° C. to heat the sensor element 201. A state is achieved in which the aforementioned pump cells 21, 41, 50 are controlled by the controller 96, and the voltages V0, V1, V2, Vref are obtained from the aforementioned sensor cells 80 to 83. A state is achieved in which the reference-gas adjustment pump cell 90 is not controlled by the controller 96. In this state, as a measurement-object gas, a gas simulating an exhaust gas in a lean state is passed through a pipe, and subsequently, a gas simulating an exhaust gas in a rich state is passed through the pipe, thus switching of the measurement-object gas from a lean state to a rich state was simulated. The voltage Vref then was continuously measured, and the manner of temporal change in the voltage Vref was studied. Similarly, also for Example 3, the manner of temporal change in the voltage Vref was studied.

Specifically, when the gas to be passed through the pipe is switched from a lean state to a rich state, the voltage Vref rose in each of Examples 2, 3. The value of the voltage Vref immediately before rise thereof is assumed to be 0%, the value of the voltage Vref after being stabilized after the rise is assumed to be 100%, and the response time [msec] of the voltage Vref is defined by the time required for the voltage Vref to change from 10% to 90%. A shorter response time indicates a higher responsiveness of the voltage Vref. The response time in Example 2 was 380 msec, and the response time in Example 3 was 400 msec. From this result, it was verified that the responsiveness of rising of the voltage Vref is higher in Example 2 in which the pump outer electrode 23p and the voltage outer electrode 23s are both provided than in Example 3 in which the outer pump electrode 923 is disposed instead of these electrodes. The responsiveness of falling of the voltage Vref at the time of switching the gas to be passed through the pipe from a rich state to a lean state was studied in the same manner, and the responsiveness was higher in Example 2 than in Example 3.

Next, in a state where the gas sensor 200 in Example 2 was placed in the atmosphere, a continuous test in atmosphere was conducted in the same manner as described above, that is, the sensor element 201 was driven by the controller 96 to operate until 500 hours elapsed. For the gas sensor in Example 3, a continuous test in atmosphere was also conducted in the same manner. The atmosphere is higher in oxygen concentration than the exhaust gas, and the noble metal in the electrode is likely to be oxidized and deteriorated, thus the continuous test in atmosphere is an accelerated deterioration test for electrode. For Examples 2, 3 after the continuous test in atmosphere was conducted, the response time [msec] of the voltage Vref was measured by the aforementioned method.

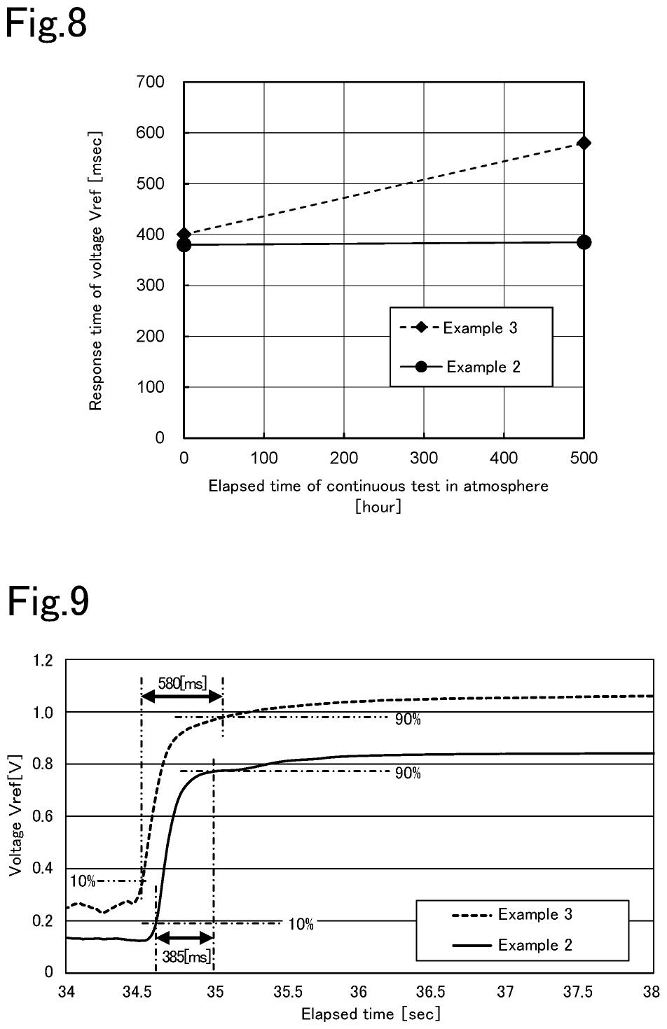

FIG. 8 shows graphs illustrating the change in response time of the voltage Vref before and after the continuous test in atmosphere in Examples 2, 3. As illustrated in FIG. 8, in Example 3, the response time (580 msec) after the continuous test in atmosphere (elapsed time is 500 hours) is longer than the response time (400 msec) before the continuous test in atmosphere (elapsed time is 0 hour), that is, the responsiveness has deteriorated. In contrast, in Example 2, the response time changed from 380 msec to 385 msec only before and after the continuous test in atmosphere, thus change in the response time was little. From this result, it was verified that deterioration of the response time of the voltage Vref with use of the gas sensor is further reduced in Example 2 in which the pump outer electrode 23p and the voltage outer electrode 23s are both provided than in Example 3 in which the outer pump electrode 923 is disposed instead of these electrodes. FIG. 9 shows graphs illustrating the manner of temporal change in the voltage Vref in Examples 2, 3 after the continuous test in atmosphere. In FIG. 9, the voltages Vref corresponding to 10% and 90% are shown for each of Examples 2, 3, where the value of the voltage Vref immediately before rise thereof is assumed to be 0%, and the value of the voltage Vref after being stabilized after the rise is assumed to be 100%. In addition, in FIG. 9, the value of the aforementioned response time was shown for each of Examples 2, 3, where the response time was measured as the time required for the voltage Vref to change from 10% to 90%.

Note that the sensor element in Example 3 has substantially the same configuration as that of the sensor element 101. Not only in Example 2 but also in Example 3, the pump reference electrode 42p and the voltage reference electrode 42s are provided, thus, the same effect as that of the gas sensor 100 of the above-described first embodiment is achieved. Therefore, Example 3 is not a comparative example, and corresponds to an example of the present invention.

When the controller 96 detects the oxygen concentration in the measurement-object gas outside the sensor element 201 based on the voltage Vref of the Vref detection sensor cell 83, as a kind of detection of the oxygen concentration, whether the measurement-object gas outside the sensor element 201 is in a rich state or a lean state may be determined based on the voltage Vref. For example, a predetermined threshold to determine whether the voltage Vref is in a rising state or a falling state is pre-stored in the storage unit 98, and the controller 96 may binarize an obtained voltage Vref based on the threshold to determine whether the measurement-object gas is in a rich state or a lean state. In this manner, the gas sensor 200 functions not only as an NOx sensor but also as a lambda sensor (air-fuel ratio sensor). Note that in the gas sensor 100 in the first embodiment also, the controller 96 may determine whether the measurement-object gas is in a rich state or a lean state in the same manner as described above.

Of the correspondence relationships between the components in this embodiment and the components in the present invention, particularly, the correspondence relationships different from those in the first embodiment will now be clarified. The first internal cavity 20 in this embodiment corresponds to an oxygen concentration adjustment chamber of the present invention, the pump outer electrode 23p corresponds to a pump outer electrode, the main pump cell 21 corresponds to an adjustment chamber pump cell, the voltage outer electrode 23s corresponds to a voltage outer electrode, and the Vref detection sensor cell 83 corresponds to an outer sensor cell. In addition, the controller 96 corresponds to an adjustment chamber pump cell controller and an oxygen concentration detector.

In the gas sensor 200 in this embodiment described in detail above, the pump outer electrode 23p and the voltage outer electrode 23s are separately provided outside the sensor element 201. Accordingly, the pump currents Ip0 to Ip3 do not flow through the voltage outer electrode 23s, thus the voltage Vref of the Vref detection sensor cell 83 does not include a voltage drop of the voltage outer electrode 23s due to the pump currents Ip0 to Ip3. Consequently, the voltage Vref has a value which corresponds with higher accuracy to the oxygen concentration in the measurement-object gas outside the sensor element 201, thus the accuracy of detection of the oxygen concentration in the measurement-object gas using the Vref detection sensor cell 83 is improved. The voltage Vref is the voltage across the voltage outer electrode 23s and the voltage reference electrode 42s, and the pump currents Ip0 to Ip3 do not flow through each of the voltage outer electrode 23s and the voltage reference electrode 42s. Thus, the voltage Vref has a value which corresponds with higher accuracy to the oxygen concentration in the measurement-object gas outside the sensor element 201.

The controller 96 causes the main pump cell 21 to pump out oxygen from the first internal cavity 20 or pump oxygen into the first internal cavity 20 by controlling the main pump cell 21 so that the oxygen concentration in the first internal cavity 20 reaches a predetermined low concentration. In this case, the direction of the pump current Ip0 which flows through the main pump cell 21 may be switched to the reverse direction. However, since the pump outer electrode 23p and the voltage outer electrode 23s are separately provided in the sensor element 201, the voltage Vref is not affected by the time required for change in the pump current Ip0. Consequently, when the oxygen concentration in the measurement-object gas is switched between a high state in which the oxygen concentration is higher than a predetermined low concentration and a low state, the responsiveness of the voltage Vref is not likely to reduce.

The present invention is not limited whatsoever to the above embodiments, and various embodiments are possible so long as they belong within the technical scope of the present invention.

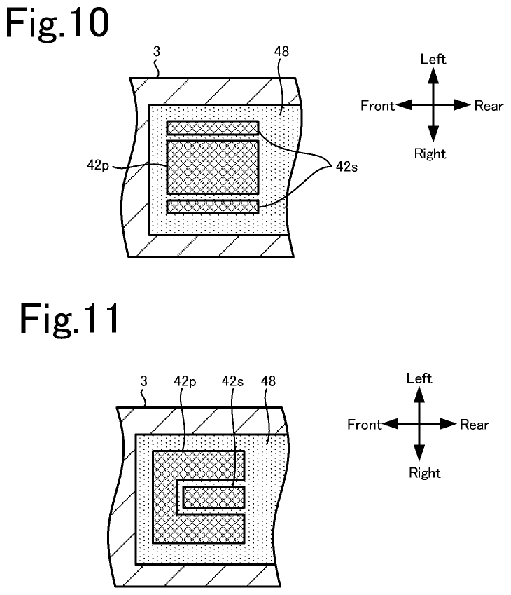

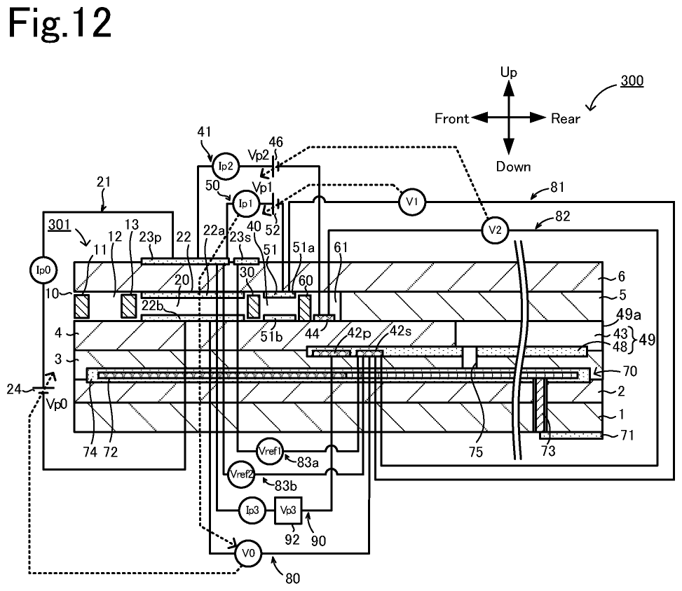

For example, in the first and second embodiments described above, the pump reference electrode 42p and the voltage reference electrode 42s are disposed side by side in the front-rear direction, however, may be disposed side by side in the left-right direction. As illustrated in FIG. 10, the voltage reference electrode 42s may be disposed at each of the right and left sides of the pump reference electrode 42p. The two voltage reference electrodes 42s illustrated in FIG. 10 are electrically connected by a lead wire which is not illustrated, and function as one voltage reference electrode. As illustrated in FIG. 11, the pump reference electrode 42p may have a recessed portion, and the voltage reference electrode 42s may be disposed in the recessed portion. In this manner, the voltage reference electrode 42s is surrounded by the pump reference electrode 42p in three directions, that is, the front and left-right directions, thus the oxygen pumped into the periphery of the pump reference electrode 42p is likely to reach the periphery of the voltage reference electrode 42s.

The aforementioned various aspects of the pump reference electrode 42p and the voltage reference electrode 42s in FIGS. 2, 10, 11 may be applied to the aspects of the pump outer electrode 23p and the voltage outer electrode 23s. However, the pump outer electrode 23p and the voltage outer electrode 23s do not need to be disposed close to each other. It is preferable that the pump outer electrode 23p and the voltage outer electrode 23s be disposed with a certain gap therebetween so that the voltage Vref does not change due to the effect of the oxygen pumped out into the periphery of the pump outer electrode 23p.

In the above-described first embodiment, it has been explained that the voltage reference electrode 42s is preferably reduced in area to lower the thermal electromotive force. Similarly, the voltage outer electrode 23s is preferably reduced in area to lower the thermal electromotive force.

In the above-described first embodiment, the fourth diffusion control section 60 is formed as a slit-shaped gap, but is not limited thereto. The fourth diffusion control section 60 may be formed as a porous body (e.g., a ceramic porous body such as alumina (Al₂O₃)). For example, the fourth diffusion control section 60 formed as a porous body may cover the measurement electrode 44. In this case, the periphery of the measurement electrode 44 functions as a measurement chamber. In other words, the periphery of the measurement electrode 44 serves the same function as the third internal cavity 61.

In the above-described second embodiment, the controller 96 may obtain not only the voltage Vref across the voltage outer electrode 23s and the voltage reference electrode 42s, but also the voltage across the pump outer electrode 23p and the voltage reference electrode 42s. FIG. 12 is a schematic cross-sectional view of a gas sensor 300 according to a modification. A sensor element 301 of the gas sensor 300 includes a Vref1 detection sensor cell 83a and a Vref2 detection sensor cell 83b. The Vref1 detection sensor cell 83a is the same sensor cell as the Vref detection sensor cell 83 of the sensor element 201. In the Vref1 detection sensor cell 83a, a voltage Vref1 is generated between the voltage outer electrode 23s and the voltage reference electrode 42s. The Vref2 detection sensor cell 83b is an electrochemical sensor cell including: the second solid electrolyte layer 6, the spacer layer 5, the first solid electrolyte layer 4, the third substrate layer 3, the pump outer electrode 23p, and the voltage reference electrode 42s. In the Vref2 detection sensor cell 83b, a voltage Vref2 is generated between the pump outer electrode 23p and the voltage reference electrode 42s. The gas sensor 300 can determine whether the pump outer electrode 23p is deteriorated based on the difference between the voltage Vref1 and the voltage Vref2. For example, the controller 96 obtains a current Ip4 (e.g., the total value of the pump currents Ip0 to Ip3) which flows through the pump outer electrode 23p, the voltage Vref1 and the voltage Vref2 at a predetermined deterioration determination timing, and calculates the difference Da between the voltage Vref1 and the voltage Vref2 obtained. Next, the controller 96 calculates a reference value for the difference between the voltage Vref1 and the voltage Vref2 based on the obtained current Ip4. The reference value is a value corresponding to the difference between the voltage Vref1 and the voltage Vref2 in a state where the pump outer electrode 23p is not deteriorated. The difference between the voltage Vref1 and the voltage Vref2 includes a voltage drop in the pump outer electrode 23p due to the current which flows through the pump outer electrode 23p, thus the controller 96 calculates a reference value based on the obtained pump current Ip4. For example, a relational expression (e.g., the expression of a linear function) and a map representing a correspondence relationship between the current Ip4 and the reference value are pre-stored in the storage unit 98, and the controller 96 calculates a reference value using the obtained current Ip4 and the correspondence relationship. Note that when the rate of the current Ip0 to the current Ip4 (the total value of the currents Ip0 to Ip3) is high, a reference value may be calculated based on the current Ip0 rather than the current Ip4. It is determined whether the pump outer electrode 23p is deteriorated based on whether the difference Da deviates from the reference value (e.g., whether the difference between the difference Da and the reference value exceeds a predetermined threshold). The pump currents Ip0 to Ip3 flow through the pump outer electrode 23p with use of the sensor element 301, thus the pump outer electrode 23p deteriorates. Thus, even when the current which flows through the pump outer electrode 23p is in the same state as before the deterioration, the voltage drop in the pump outer electrode 23p due to the current flow is increased than before the deterioration. Thus, the difference Da between the voltage Vref1 and the voltage Vref2 tends to increase as the pump outer electrode 23p deteriorates. Therefore, the controller 96 can determine whether the pump outer electrode 23p is deteriorated by comparing the difference Da with the aforementioned reference value. When the pump outer electrode 23p deteriorates, the accuracy of measurement of the NOx concentration may be reduced by a change in the values of the pump currents Ip0 to Ip3 which are caused to flow by respective voltages Vp0 to Vp3. When the controller 96 is able to determine deterioration of the pump outer electrode 23p, for example, the controller 96 can prevent the accuracy of measurement of the NOx concentration from remaining at a low level through handling such as transmission of error information to an engine ECU. Note that the controller 96 can determine not only whether the pump outer electrode 23p is deteriorated, but also the degree of deterioration of the pump outer electrode 23p based on the magnitude of the difference Da, or based on the degree of deviation (e.g., the magnitude of the difference between the difference Da and the reference value) between the difference Da and the reference value. In addition, the controller 96 may change control of the sensor element 301 so that effect of deterioration is canceled according to presence or absence of deterioration or the degree of deterioration of the pump outer electrode 23p. For example, the controller 96 may change at least one of the aforementioned target values V0*, V1*, V2*, and Ip1* based on the difference Da or based on the difference between the difference Da and the reference value. Alternatively, the controller 96 may change the amount of oxygen pumped into the periphery of the pump reference electrode 42p by changing the voltage Vp3 to change the pump current Ip3 based on the difference Da or based on the difference between the difference Da and the reference value.

In the above-described first embodiment, the reference-gas adjustment pump cell 90 includes the outer pump electrode 23 disposed outside the element body as a pumping-in source electrode which serves as a source to pump oxygen into the reference-gas introduction portion 49. Similarly, in the above-described second embodiment, as the pumping-in source electrode, the pump outer electrode 23p disposed outside the element body is provided. However, without being limited to this, the pumping-in source electrode may be disposed inside or outside the element body so as to be in contact with the measurement-object gas. For example, the inner pump electrode 22 in FIG. 1 may be used as a pumping-in source electrode, and the reference-gas adjustment pump cell 90 may pump oxygen into the reference-gas introduction portion 49 from the periphery of the inner pump electrode 22. The reference-gas adjustment pump cell 90 may pump out oxygen from the periphery of the pump reference electrode 42p.

In the above-described first embodiment, the element body of the sensor element 101 is a layered body having a plurality of solid electrolyte layers (layers 1 to 6), but is not limited thereto. The element body of the sensor element 101 may include at least one oxygen-ion-conductive solid electrolyte layer, and may be internally provided with a measurement-object gas flow portion. For example, in FIG. 1, the layers 1 to 5 other than the second solid electrolyte layer 6 may be structural layers (e.g., layers composed of alumina) composed of a material other than that of solid electrolyte layers. In this case, the electrodes possessed by the sensor element 101 may be disposed in the second solid electrolyte layer 6. For example, the measurement electrode 44 in FIG. 1 may be disposed on the lower surface of the second solid electrolyte layer 6. Also, the reference-gas introduction space 43 may be provided in the spacer layer 5 instead of the first solid electrolyte layer 4, the reference-gas introduction layer 48 may be provided between the second solid electrolyte layer 6 and the spacer layer 5 instead of between the first solid electrolyte layer 4 and the third substrate layer 3, and the pump reference electrode 42p and the voltage reference electrode 42s may be provided rearward of the third internal cavity 61 and on the lower surface of the second solid electrolyte layer 6. The same applies to the second embodiment.

In the above-described first to second embodiments, the controller 96 sets (feedback-controls) the target value V0* of the voltage V0 based on the pump current Ip1 so that the pump current Ip1 reaches the target value Ip1*, and the controller 96 feedback-controls the voltage Vp0 so that the voltage V0 reaches the target value V0*, but may perform another control. For example, the controller 96 may feedback-control the voltage Vp0 based on the pump current Ip1 so that the pump current Ip1 reaches the target value Ip1*. In other words, the controller 96 may directly control the voltage Vp0 (eventually control the pump current IpC) based on the pump current Ip1 without obtaining the voltage V0 from the V0 detection sensor cell 80 and setting the target value V0*. Also, in this situation, the controller 96 feedback-controls the voltage Vp1 so that the voltage V1 reaches the target value V1*, thus the controller 96 controls the oxygen concentration in the first internal cavity 20 upstream of the second internal cavity 40 at a predetermined low concentration using the main pump cell 21 so that the pump current Ip1 reaches the target value Ip1* and the oxygen concentration in the second internal cavity 40 reaches a predetermined low concentration (an oxygen concentration corresponding to the voltage V1). Therefore, even when control according to such a modification is performed, as in the description of the second embodiment, when the oxygen concentration in the measurement-object gas is switched between a high state in which the oxygen concentration is higher than a predetermined low concentration and a low state, the direction of the pump current Ip0 is switched to the reverse direction. Thus, even when control according to such a modification is performed, the effect of preventing reduced responsiveness of the voltage Vref is obtained as in the second embodiment described above by separately providing the pump outer electrode 23p and the voltage outer electrode 23s as in the second embodiment.

In the above-described first embodiment, the oxygen concentration adjustment chamber has the first internal cavity 20 and the second internal cavity 40, however, without being limited to this, for example, the oxygen concentration adjustment chamber may include a still another internal cavity, or one of the first internal cavity 20 and the second internal cavity 40 may be omitted. Similarly, in the above-described first embodiment, the adjustment pump cell has the main pump cell 21 and the auxiliary pump cell 50, however, without being limited to this, for example, the adjustment pump cell may include a still another pump cell, and one of the main pump cell 21 and the auxiliary pump cell 50 may be omitted. For example, when the oxygen concentration in the measurement-object gas can be sufficiently reduced to a low oxygen concentration only by the main pump cell 21, the auxiliary pump cell 50 may be omitted. When the auxiliary pump cell 50 is omitted, the controller 96 may omit the aforementioned setting of the target value V0* based on the pump current Ip1. Specifically, a predetermined target value V0* is pre-stored in the storage unit 98, and the controller 96 may control the main pump cell 21 by feedback-controlling the voltage Vp0 of the variable power supply 24 so that the voltage V0 reaches the target value V0*. The same applies to the second embodiment.

In the above-described first embodiment, the gas sensor 100 detects the NOx concentration as a specific gas concentration, however, without being limited to this, another oxide concentration may be used as a specific gas concentration. In the case where the specific gas is an oxide, when the specific gas itself is reduced in the third internal cavity 61, oxygen is produced as in the above-described first embodiment, thus the controller 96 can detect a specific gas concentration based on the detection value according to the oxygen. Alternatively, the specific gas may be a non-oxide such as ammonia. In the case where the specific gas is a non-oxide, when the specific gas is converted to an oxide (e.g., ammonia is oxidized and converted to NO), for example, in the first internal cavity 20, and the converted oxide is reduced in the third internal cavity 61, oxygen is produced, thus the controller 96 can obtain a detection value according to the oxygen and detect a specific gas concentration. In this manner, regardless of whether the specific gas is an oxide or a non-oxide, the gas sensor 100 can detect a specific gas concentration based on the oxygen produced from the specific gas in the third internal cavity 61. The same applies to the second embodiment.

What is claimed is:

1. A sensor element for detecting a specific gas concentration in a measurement-object gas, the sensor element comprising:
an element body including an oxygen-ion-conductive solid electrolyte layer and internally provided with a measurement-object gas flow portion that introduces a measurement-object gas and causes the measurement-object gas to flow therethrough;
a reference-gas introduction portion disposed inside the element body, the reference-gas introduction portion being configured to introduce a reference gas serving as a reference for detecting a specific gas concentration in the measurement-object gas;
a reference-gas adjustment pump cell having a pump reference electrode disposed inside the element body so as to be in contact with the reference gas introduced to the reference-gas introduction portion, the reference-gas adjustment pump cell being configured to pump oxygen into a periphery of the pump reference electrode; and
a sensor cell having a voltage reference electrode disposed inside the element body so as to be in contact with the reference gas introduced to the reference-gas introduction portion, and a measurement-object gas-side electrode disposed inside or outside the element body so as to be in contact with the measurement-object gas, the sensor cell being configured to generate a voltage based on an oxygen concentration in a periphery of the measurement-object gas-side electrode.

2. The sensor element according to claim 1, further comprising
a measurement pump cell that pumps out oxygen produced from the specific gas in a measurement chamber of the measurement-object gas flow portion,
wherein the measurement-object gas-side electrode is a measurement electrode disposed in the measurement chamber, and
the sensor cell is a measurement sensor cell that generates a voltage based on an oxygen concentration in the measurement chamber.

3. The sensor element according to claim 2, further comprising:
an adjustment chamber pump cell having an adjustment electrode disposed in an oxygen concentration adjustment chamber upstream of the measurement chamber of the measurement-object gas flow portion, and a pump outer electrode disposed outside the element body, the adjustment chamber pump cell being configured to pump oxygen out from the oxygen concentration adjustment chamber or pump oxygen into the oxygen concentration adjustment chamber; and
an outer sensor cell having a voltage outer electrode disposed outside the element body, and the voltage reference electrode, the outer sensor cell being configured to generate a voltage based on an oxygen concentration in the measurement-object gas outside the element body.

4. A gas sensor comprising:
the sensor element according to claim 2; and
a measurement pump cell controller that causes the measurement pump cell to pump out oxygen from the measurement chamber by feedback-controlling the measurement pump cell so that the voltage of the measurement sensor cell reaches a target voltage.

5. The gas sensor according to claim 4, further comprising
a reference-gas adjustment unit that causes the reference-gas adjustment pump cell to pump oxygen into the periphery of the pump reference electrode by applying a repeatedly turned ON/OFF control voltage to the reference-gas adjustment pump cell,
wherein the measurement pump cell controller obtains the voltage of the measurement sensor cell in a period when the repeatedly turned ON/OFF control voltage is OFF, and feedback-controls the measurement pump cell so that the obtained voltage reaches the target voltage.

6. A gas sensor comprising:
the sensor element according to claim 3;
an adjustment chamber pump cell controller that causes the adjustment chamber pump cell to pump out oxygen from the oxygen concentration adjustment chamber or pump oxygen into the oxygen concentration adjustment chamber by controlling the adjustment chamber pump cell so that an oxygen concentration in the oxygen concentration adjustment chamber reaches a predetermined low concentration; and
an oxygen concentration detector that detects an oxygen concentration in the measurement-object gas outside the element body based on the voltage of the outer sensor cell.

7. A gas sensor comprising:
the sensor element according to claim 3; and
a measurement pump cell controller that causes the measurement pump cell to pump out oxygen from the measurement chamber by feedback-controlling the measurement pump cell so that the voltage of the measurement sensor cell reaches a target voltage.

8. The gas sensor according to claim 7, further comprising
a reference-gas adjustment unit that causes the reference-gas adjustment pump cell to pump oxygen into the periphery of the pump reference electrode by applying a repeatedly turned ON/OFF control voltage to the reference-gas adjustment pump cell,
wherein the measurement pump cell controller obtains the voltage of the measurement sensor cell in a period when the repeatedly turned ON/OFF control voltage is OFF, and feedback-controls the measurement pump cell so that the obtained voltage reaches the target voltage.
